SEQUENCE LISTING (9 sequences)

SEQ ID NO: 1 - DNA, 29 bp, Artificial Sequence
  lacIq promoter from pQE80
gtgcaaaacctttcgcggtatggcatgat

SEQ ID NO: 2 - DNA, 29 bp, Escherichia coli
gcgcaaaacctttcgcggtatggcatgat

SEQ ID NO: 3 - protein, 20 aa, Artificial Sequence
  N-terminal maltose binding protein fusion
SHHHHHHHHASENLYFQAIA

SEQ ID NO: 4 - protein, 8 aa, Human rhinovirus
LEVLFQGP

SEQ ID NO: 5 - protein, 6 aa, Artificial Sequence
  tetraCys motif
CCPGCC

SEQ ID NO: 6 - protein, 9 aa, Artificial Sequence
  Amino acid sequence required for the attB1 site of Gateway cloning
TSLYKKAGS

SEQ ID NO: 7 - protein, 7 aa, Artificial Sequence
  TEV protease recognition site
ENLYFQS

SEQ ID NO: 8 - protein, 24 aa, Artificial Sequence
  N-terminal sequence of Tm3CP substrate protein
GPCCPGCCTSLYKKAGSENLYFQS

SEQ ID NO: 9 - protein, 23 aa, Artificial Sequence
  Fluorescently labeled peptide released by proteolysis
GPCCPGCCTSLYKKAGSENLYFQ

(19) United States
(12) Patent Application Publication
(10) Pub. No.: US 2008/0286749 A1
(43) Pub. Date: Nov. 20, 2008

(54) ENHANCED PROTEIN EXPRESSION USING AUTO-INDUCTION MEDIA

(76) Inventors: Brian G. Fox, Madison, WI (US); Paul G. Blommel, Oregon, WI (US)

Correspondence Address: WARF/BHGL, P.O. Box 10395, Chicago, IL, 60610, US

(21) Appl. No.: 12/100,925

(22) Filed: Apr. 10, 2008

Related U.S. Application Data
(60) Provisional application No. 60/923,104, filed on Apr. 12, 2007.

(51) Int. Cl. C12N 1/20; C12Q 3/00; C12N 15/70

(52) U.S. Cl. 435/3; 435/253.6; 435/471

(57) ABSTRACT

Methods for refining the compositions of bacterial growth media to improve heterologous expression of desired recombinant target genes are provided. Also provided are compositions and culture media obtained using the above methods.

### CROSS-REFERENCE TO RELATED APPLICATIONS

This invention claims priority to U.S. Provisional Patent Application Ser. No. 60/923,104, filed Apr. 12, 2007, which is herein incorporated by reference.

### GOVERNMENT INTERESTS

This invention was made with United States government support awarded by the National Institutes of Health, grant NIH 1U54 GM074901. The United States government has certain rights in this invention.

### FIELD OF THE INVENTION

The present invention relates generally to the field of cell growth and culture. More particularly, the present invention provides novel methods and compositions for the growth of cells in order to improve expression of recombinant target genes.

### BACKGROUND

Recombinant DNA technology makes it possible to clone desired coding sequences into expression vectors that can direct the production of the corresponding proteins in suitable host cells. The resulting proteins are widely useful, as objects of biochemical, biophysical, structural and functional studies for understanding basic biological processes, as enzymes to serve as research tools or produce valuable chemicals, as diagnostics, vaccines, therapeutics or targets for developing medically useful drugs, or for protein chips, to mention a few. Reliable and reproducible methods for high throughput production of proteins are required for structural genomics, functional proteomics, drug discovery and other current protein biochemistry and enzymology initiatives.

As one approach to this problem, the auto-induction method has been used for production of recombinant proteins in E. coli (Studier, 2005, Protein Expr. Purif. 41: 207-234; U.S. Patent Application No. 2004/0180423 A1). Auto-induction of transcription of cloned DNA in cultures of bacterial cells is an approach that employs different carbon sources to support cell growth and protein expression without the requirement to monitor the culture growth state. Auto-induction arises from a complex set of changes in growth conditions and host regulatory responses.

Auto-induction protocols were originally formulated for T7 promoter-based expression, and are based on the function of lac operon regulatory elements in mixtures of glucose, glycerol and lactose under diauxic growth conditions. During the initial growth period, glucose is preferentially used as a carbon source and protein expression is low due to catabolite repression of alternative carbon utilization pathways and binding interactions between lac repressors (LacI) and lac operators (lacO). As glucose is depleted, catabolite repression is relieved, leading to a shift in cellular metabolism toward the import and consumption of lactose and glycerol. Lactose import results in the production of allolactose from lactose by a reaction of β-galactosidase. Allolactose then acts as the physiological inducer of the lac operon.

An inducible T7 expression system is highly effective and is used for production of proteins from cloned coding sequences in the bacterium Escherichia coli. IPTG (isopropyl-beta-D-thiogalactopyranoside) has typically been used to induce expression of target proteins in the inducible T7 expression system. Lactose will also cause induction and, being much cheaper than IPTG, may be preferable for large-scale production (Hoffman et al., 1995, Protein Express. Purif. 6: 646-654). A problem in using inducible T7 expression systems is that T7 RNA polymerase is so active that a small basal level can lead to a substantial expression of target protein even in the absence of added inducer. Cultures growing in certain complex media induce the target protein to high levels upon approach to saturation even when the T7 lac promoter is used.

Several factors complicate the use of auto-induction. Since multiple carbon sources are present in the auto-induction medium, their relative amounts and their patterns of usage are critically important contributors to the outcome of the auto-induction expression. Furthermore, for optimal utility, the auto-induction method should be easy to perform in both small-scale screening and large-scale production and should also provide correlation between the results obtained at the different scales of operation. However, this scaling requirement introduces variability arising from physical parameters such as the extent of aeration associated with different vessels used for cell culture. Indeed, the availability of O₂ can profoundly affect the outcome of auto-induction experiments, but the origin of this effect is not clear.

For recombinant expression systems that operate under control of the lac operon, the appearance of allolactose during auto-induction initiates the expression of heterologous proteins. However, the construction of recombinant expression systems makes the circumstances of induction more complicated than in wild-type E. coli. For example, E. coli cells harboring a multi-copy expression plasmid may produce LacI at levels 200-fold higher than that present in wild-type cells. Currently, there is limited experimental information on the diauxic behavior of cells expressing high concentrations of LacI (Chen et al., 1991, Biotechnol. Bioeng. 38: 679-687).

Auto-induction protocols could be attractive for both small and large-scale growth of bacterial cultures due to the reduced requirement for process monitoring and the higher achievable cell density compared to traditional IPTG induction. However, protein expression in small-scale screening auto-induction medium was often found to be drastically lower than that obtained from large-scale culture (Sreenath et al., 2005, Protein Express. Purif. 40: 256-267). Thus, because of issues in non-reproducibility of small-scale screening for heterologous expression and large-scale production of the desired recombinant proteins, the auto-induction method has not been uniformly adopted within the NIH-funded Protein Structure Initiative.

Given the importance of bacterial protein expression studies, it is important to more fully understand the underlying metabolic and physical constraints to reproducibility and productivity of auto-induction approaches. In protein expression, it may be desirable to attain high levels of induced protein expression while having low levels of basal protein expression. There is a need for a bacterial growth medium that will reproducibly improve heterologous expression of recombinant genes.

### BRIEF SUMMARY

Methods are provided for designing culture media that promote induction of transcription of heterologous DNA in cultures of bacterial cells, which include: a) providing bacterial cells comprising recombinant expression vectors comprising the heterologous DNA operably connected to a promoter whose activity can be induced by one or more constituents of the culture medium; b) defining a first medium constituent; c) changing the concentration of the medium constituent in the culture medium; d) evaluating the outcome of the change in the concentration of the medium constituent to determine the change that gives the most favorable result for expression of heterologous DNA; e) adopting the changed concentration of the medium constituent that gives the most favorable result as a new starting condition; f) defining a next medium constituent; and g) repeating steps c) to e) with a different medium constituent, to determine a new more favorable composition of the culture medium for promoting transcription of the heterologous DNA. Changing the concentration of the constituents may include increasing or decreasing the concentration of the constituents in the culture medium. The medium constituents may include one or more carbon sources selected from the group consisting of glucose, lactose, glycerol, rhamnose, arabinose, succinate, fumarate, malate, citrate, acetate, maltose and sorbitol. The medium constituents may include a pH buffering compound, which may be dicarboxylic acid. The dicarboxylic acid may be selected from the group consisting of oxalic acid, aspartic acid, fumaric acid, glutamic acid, succinic acid, malonic acid, glutaric acid, phthalic acid.

The methods may be practiced with bacterial cells, for example Escherichia coli cells. The bacterial cells may be grown batchwise. The ability to induce the promoter may be dependent on the metabolic state of the bacterial cells. In one example, the promoters may be selected from the group consisting of lac promoters, T7 promoters, T7/lac promoters, T5 promoters, or T5/lac promoters. In one example, the promoter may be repressed by a lac repressor.

In the practice of the methods, the culture media may include from about 0.01% w/v to about 0.02% w/v of glucose.

Culture media are provided, which are obtained using the methods of the present invention. In one example, the culture media may include from about 0.01% w/v to about 0.02% w/v of glucose. In another example, the culture media may include from about 0.4% w/v to about 0.6% w/v of lactose. In another example, the culture media may include from about 0.7% w/v to about 0.9% w/v of glycerol. In yet another example, the culture media may include from about 0.35% w/v to about 0.40% w/v of dicarboxylic acid. In one embodiment, the culture media may include about 0.01% w/v to about 0.02% w/v of glucose, about 0.4% w/v to about 0.6% w/v of lactose, about 0.7% w/v to about 0.9% w/v of glycerol, and about 0.35% w/v to about 0.40% w/v of dicarboxylic acid.

Methods are provided for promoting auto-induction of transcription of heterologous DNA in cultures of bacterial cells, which include: a) providing bacterial cells comprising a recombinant expression vector comprising heterologous DNA operably connected to a promoter whose activity can be induced by an exogenous inducer; b) providing culture medium that includes culture medium comprising about 0.001% w/v to about 0.5% w/v of glucose, about 0.01% w/v to about 3% w/v of lactose, and about 0.1% w/v to about 5% w/v of glycerol; and c) growing the bacterial cells in the culture media to express heterologous DNA. Changing the concentration of the constituents may include increasing or decreasing the concentration of the constituents in the culture medium. In some embodiments, the culture media may include one or more carbon sources selected from the group consisting of glucose, lactose, glycerol, rhamnose, arabinose, succinate, fumarate, malate, citrate, acetate, maltose and sorbitol. The culture media may include a pH buffering compound, which may be dicarboxylic acid. The culture media may further include between about 0.05% w/v to about 4% w/v of dicarboxylic acid. The dicarboxylic acid may be selected from the group consisting of oxalic acid, aspartic acid, fumaric acid, glutamic acid, succinic acid, malonic acid, glutaric acid, phthalic acid. The methods may be practiced with bacterial cells, for example Escherichia coli cells. The bacterial cells may be grown batchwise. The ability to induce the promoter may be dependent on the metabolic state of the bacterial cells. In one example, the promoters may be selected from the group consisting of lac promoters, T7 promoters, T7/lac promoters, T5 promoters, or T5/lac promoters. In one example, the promoter may be repressed by a lac repressor. In the practice of the methods, the culture medium may include from about 0.01% w/v to about 0.02% w/v of glucose. The culture medium may include from about 0.4% w/v to about 0.6% w/v of lactose. The culture medium may include from about 0.7% w/v to about 0.9% w/v of glycerol. The culture medium may include from about 0.35% w/v to about 0.40% w/v of dicarboxylic acid. In one embodiment of the practice of the methods, the culture medium may include about 0.001% w/v to about 0.5% w/v of glucose, about 0.01% w/v to about 3% w/v of lactose, and about 0.1% w/v to about 5% w/v of glycerol. The culture medium may further include about 0.05% w/v to about 4% w/v of dicarboxylic acid. In another embodiment of the practice of the methods, the culture medium may include about 0.01% w/v to about 0.02% w/v of glucose, about 0.4% w/v to about 0.6% w/v of lactose, and about 0.7% w/v to about 0.9% w/v of glycerol. The culture medium may further include about 0.05% w/v to about 4% w/v of dicarboxylic acid.

### BRIEF DESCRIPTION OF THE DRAWINGS

FIG. 1 is a schematic representation of the experimental space for single step factorial change (increase, no change, decrease) of three carbon sources (glycerol, glucose, and lactose in this example), with the starting concentration point shown as a dark sphere in the center of the cube.

FIG. 2 illustrates a restriction map of a T5/lac2 expression vector.

FIG. 3 is a graph of basal protein expression levels of luciferase in different strains, under catabolite repressed conditions.

FIG. 4 depicts images of auto-induction expression results from small and large scale production of four target proteins, shown by SDS-PAGE, using original media as defined by Studier, 2005, Protein Expr. Purif. 41: 207-234 (top panels), and evolved media modified according to this invention (bottom panels).

FIG. 5 shows graphs of the patterns of carbon utilization for glycerol (dark filled squares) and lactose (gray filled circles) in the context of T7 promoter expression system (left panels) and a T5/lac2 expression system (right panels).

FIG. 6 shows graphs of the patterns of carbon source consumption for glycerol (dark filled squares) and arabinose (gray filled circles) in the context of using arabinose as an inducer (left panels) and using rhamnose as an inducer (right panels).

FIG. 7 depicts images of SDS-PAGE demonstration of scale dependence during auto-induction.

FIG. 8 illustrates restriction maps of expression plasmids useful for practicing the invention.

FIG. 9 shows graphs of response surfaces arising from factorial design changes in the composition of auto-induction medium and changes in LacI dosing.

FIG. 10 shows an image of SDS-PAGE analysis of eGFP expression from T5-lacI-eGFP.

FIG. 11 shows graphs of LabChip90 protein electropherograms (plots of fluorescence units over time) showing luciferase expression from the indicated luciferase expression plasmids.

FIG. 12 shows graphs of dissolved O₂ (solid lines) and pH (dashed lines) profiles for aerobic (top) and O₂-limited (bottom) growth of E. coli B834 T7-Luc completed in a Sixfors instrumented fermenter.

FIG. 13 shows graphs of HPLC determination of carbon source levels and carbon consumption patterns during the time course of O₂-limited auto-induction in E. coli B834 (DE3) transformed with T7-Luc.

FIG. 14 shows graphs of the timing of lactose consumption as a consequence of LacI dosing.

FIG. 15 shows graphs of the effect of aeration on lactose consumption with the T5-lacI-Luc expression plasmid.

FIG. 16 is a graph showing comparison of modeled expression levels for T5-lacI (solid line), T7-lacI (pET32, dashed line), T5-lacI^{q} in methionine auto-induction medium (filled diamonds) and T5-lacI^{q} in selenomethionine auto-induction medium (filled circles).

FIG. 17 is graphs depicting a topographical map that includes expression data for higher carbon source concentrations.

FIG. 18 illustrates restriction maps of three expression vectors useful for practicing this invention.

FIG. 19 is a schematic representation of the equipment used for automated two-step purification of His₇-TEV protease.

FIG. 20 is a graph depicting a representative fluorescence polarization assay of TEV protease activity present in an E. coli cell lysate.

FIG. 21 shows data on the expression of TEV protease during auto-induction from MHT238Δ in a 10-L fermenter.

FIG. 22 shows graphs with representations of the factorial experimental design experimental space.

FIG. 23 is an image of a plate containing diluted eGFP expression lysates from the media listed in Table 5 illuminated with a 340 nm light source.

### DETAILED DESCRIPTION OF THE PRESENTLY PREFERRED EMBODIMENTS

This invention relates to the field of media for growth of cells that express recombinant heterologous proteins. More particularly, the invention provides methods for refining the composition of a bacterial growth medium to improve heterologous expression of desired recombinant genes. The invention also provides culture media obtained using the above methods.

The present invention relates, in one aspect, to a method for promoting auto-induction of transcription of cloned DNA in cultures of bacterial cells, when the transcription is under the control of a promoter whose activity can be induced by an exogenous inducer. A culture media is provided which includes an inducer that causes induction of transcription from a desired promoter in genetically engineered bacterial cells, and media constituents in concentrations that are determined using the methods of the present invention. The culture medium is inoculated with a bacterial inoculum. The inoculum includes bacterial cells containing cloned DNA encoding one or more desired proteins, the transcription of which is induced by the inducer. The culture is then incubated under conditions appropriate for growth of the bacterial cells, so that the cells express the recombinant protein.

“Media constituents” refers to the constituents, i.e. ingredients of a culture medium used for growth of cells expressing recombinant heterologous proteins. Media constituents include: inorganic constituents, organic constituents, additives, hormones, promoters, etc. Examples of inorganic media constituents include carbon, hydrogen, oxygen, and other elements (e.g., N, P, S, Ca, K, Mg, Fe, Mn, Cu, Zn, B, and Mo). Examples of organic media constituents include nitrogen and carbon sources, e.g., sucrose, glucose, lactose, rhamnose, arabinose, fructose, glycerol, succinate, fumarate, malate, citrate, acetate, maltose, sorbitol, starch, or other carbohydrates, and further include dicarboxylic acids such as oxalic acid, aspartic acid, fumaric acid, glutamic acid, succinic acid, malonic acid, glutaric acid, phthalic acid, etc. Other media constituents include, e.g., casein hydrolysate, coconut milk, corn milk, malt extract, tomato juice, and yeast extract.

The present invention provides a method for producing enhanced protein expression in vitro, which takes advantage of optimization of the growth media used for growth of microorganisms that are used for expression of proteins. By “enhanced” protein expression in the foregoing context, is meant that the protein expression rate is greater in a medium conducive to growth of the microorganism, when the concentration of one or more of the medium's components is adjusted according to the methods of the present invention. By “enhanced” protein expression is also meant that the protein expression rate is greater in a medium conducive to growth of the microorganism, when the concentration of one or more of the medium's components is adjusted according to the methods of the present invention such that an inducing agent is present, or the inducing agent's concentration is optimized, than it otherwise would be under the same conditions with the inducing agent absent, or the inducing agent's concentration not optimized.

The methods of the present invention may include the preparation of culture media for the microorganisms by modifying a known microorganisms' nutrient medium using the factorial designs described herein. Alternatively, the methods may include combining a known microorganisms' nutrient medium with an inducing agent of the compositions described so as to enhance the protein expression by microorganisms in the culture medium.

In one embodiment, optimization of culture media is performed using a “factorial design” approach. Factorial design approach refers to media optimization method where certain media constituents are fixed, and other media constituents are varied in a controlled fashion (Swalley et al., 2006, Anal. Biochem. 351: 122-127; Myers and Montgomery, 2002, Response surface methodology: process and product optimization using designed experiments, 2^{nd} ed., Wiley, New York). In one exemplary embodiment, all media constituents are fixed except for glucose, glycerol and lactose, and these are then independently varied in a factorial design approach. Varying the media constituents may include: (i) keeping the concentration of particular media constituent at the same concentration as the original auto-induction media, as defined by Studier, 2005, Protein Expr. Purif. 41: 207-234; (ii) increasing the concentration of the particular media constituent relative to the concentration of the original media; or (iii) decreasing the concentration of the particular media constituent relative to the concentration of the original media. Once an optimum concentration of a particular media constituent for protein expression is determined, the concentration of that particular media constituent is held constant, and the process may be repeated with a different media constituent. The order of optimizing the concentration of particular media constituents can vary. For example, the order can be: optimizing the concentration of medium constituent 1; then optimizing the concentration of medium constituent 2; then optimizing the concentration of medium constituent 3; then optimizing the concentration of medium constituent 4; etc. Alternatively, it might be possible to optimize the concentration of particular media constituents by: optimizing the concentration of medium constituent 1; optimizing the concentration of medium constituent 2; then going back and again optimizing the concentration of medium constituent 1; etc. Alternatively, it might be possible to use any combinations of the above approaches.

The methods of the present invention may include as an additional step the use of appropriately chosen expression vectors, with promoters that can be tailored to the particular inducing agent or inducing agents used in the culture medium. Alternatively, the promoters may be tailored to be inducible by particular constituents used in the culture medium.

Particular microorganisms useful for practicing the present invention, the protein expression in which can be enhanced using the methods and compositions described herein, include bacteria, and in particular the bacterium Escherichia coli (“E. coli”).

In one example, the methods and compositions of the present invention are used to enhance the expression of TEV protease.

“Inducing agent” refers to an agent that is used to induce expression of the desired recombinant target gene. The inducing agent can, for example, be sugar, if the sugar induces expression of the desired recombinant target gene. Examples of inducing sugars include arabinose, rhamnose, lactose, and maltose. For description of the lactose induction process see, e.g., Hoffman et al., 1995, Protein Express. Purif. 6: 646-654.

“Dicarboxylic acids” are organic compounds that are substituted with two carboxylic acid functional groups. In molecular formulae for dicarboxylic acids, these groups are often written as HOOC—R—COOH, where R is usually an alkyl, alkenyl, or alkynyl group. Examples of dicarboxylic acids include oxalic acid, aspartic acid, fumaric acid, glutamic acid, succinic acid, malonic acid, glutaric acid, phthalic acid, etc.

“Diauxic” growth describes the growth phases of a bacterial colony as it metabolizes a mixture of sugars. During the first phase, cells preferentially metabolize the sugar whose catabolism is most efficient (often glucose). Only after the first sugar has been exhausted do the cells switch to the second. At the time of the “diauxic shift”, there is often a lag period during which the cell produces the enzymes needed to metabolize the second sugar.

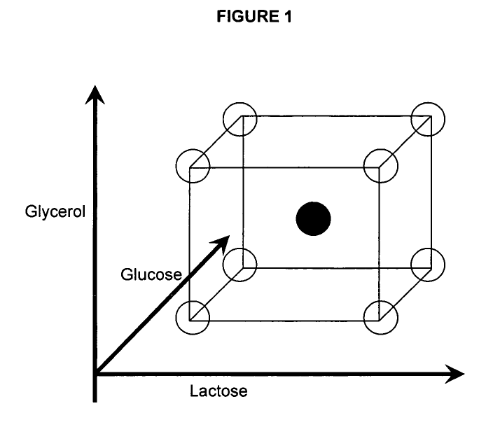

In one example, the multifactorial experimental space for determining optimal concentrations of media constituents is illustrated in FIG. 1. As shown in FIG. 1, concentrations of different carbon sources can be systematically varied as: (i) increased; (ii) no change; or (iii) decreased from the initial state. After each round of experiments, a new center point (illustrated as a dark sphere in FIG. 1) can be chosen based on the best previous result and the factorial process can be continued. Thus, in one aspect, the factorial method can define two or more constituents of the culture medium to be varied, and changes one of these constituents to low, same and high states. An experimental evaluation of the consequences is then made, which preferably includes measurement of the levels and quality of heterologous gene expression and/or heterologous protein production. The change of culture media constituents that gives the most favorable result is adopted as a new starting condition and another medium constituent is then varied through (i) low, i.e. decreased constituent concentration; (ii) same, i.e. no change in the constituent concentration; and (iii) high, i.e. increased constituent concentration states, and a new most favorable composition is determined. An example of results achieved using this factorial method is illustrated in Table 1, showing the results from approximately 60 rounds of this experimental, non-predictable evolution to modify an original starting medium for auto-induction described by Studier, 2005, Protein Expr. Purif. 41: 207-234, to one that has greater utility. The method is not limited to evaluation of carbon constituents in the media. The concentration of additional media constituents can be varied and experimentally optimized using the methods of the present invention.

A linear response model may be used to describe the consequences of the changes in the variables being studied, according to the equation:

E=C₀+C₁X₁+C₂X₂+C₃X₃+C₄X₄

where E is the measured total response, Xᵢ is the variable being changed and Cᵢ represents the partial response coefficient for that variable.

TABLE 1
Media evolution for T5/lac2 expression expressed as % (w/v)

| | Original | Final concentration in |
|---|---|---|
| Media constituents | concentration | evolved media |
| Glucose | 0.05% | 0.015% |
| Lactose | 0.2% | 0.5% |
| Glycerol | 0.5% | 0.8% |
| Dicarboxylic acid | 0.25% | 0.375% |

In one embodiment, the present invention provides for culture media that include from about 0.001% w/v to about 0.5% w/v of glucose. In another embodiment, the present invention provides for culture media that include from about 0.01% w/v to about 0.02% w/v of glucose. In yet another embodiment, the present invention provides for culture media that include about 0.015% w/v of glucose.

In one embodiment, the present invention provides for culture media that include from about 0.01% w/v to about 3% w/v of lactose. In another embodiment, the present invention provides for culture media that include from about 0.4% w/v to about 0.6% w/v of lactose. In yet another embodiment, the present invention provides for culture media that include about 0.5% w/v of lactose.

In one embodiment, the present invention provides for culture media that include from about 0.1% w/v to about 5% w/v of glycerol. In another embodiment, the present invention provides for culture media that include from about 0.7% w/v to about 0.9% w/v of glycerol. In yet another embodiment, the present invention provides for culture media that include about 0.8% w/v of glycerol.

In one embodiment, the present invention provides for culture media that include from about 0.05% w/v to about 4% w/v of dicarboxylic acid. In another embodiment, the present invention provides for culture media that include from about 0.35% w/v to about 0.40% w/v of dicarboxylic acid. In yet another embodiment, the present invention provides for culture media that include about 0.375% w/v of dicarboxylic acid.

In one embodiment, the present invention provides for culture media that include about 0.001% w/v to about 0.5% w/v of glucose, about 0.01% w/v to about 3% w/v of lactose, about 0.1% w/v to about 5% w/v of glycerol, and about 0.05% w/v to about 4% w/v of dicarboxylic acid. In another embodiment, the present invention provides for culture media that include about 0.01% w/v to about 0.02% w/v of glucose, about 0.4% w/v to about 0.6% w/v of lactose, about 0.7% w/v to about 0.9% w/v of glycerol, and about 0.35% w/v to about 0.40% w/v of dicarboxylic acid. In yet another embodiment, the present invention provides for culture media that include about 0.015% w/v of glucose, about 0.5% w/v of lactose, about 0.8% w/v of glycerol, and about 0.375% w/v of dicarboxylic acid.

In one embodiment, the present invention provides for culture media that include glucose and lactose within the ranges described above. For example, the present invention provides for culture media that include about 0.001% w/v to about 0.5% w/v of glucose, and about 0.01% w/v to about 3% w/v of lactose.

In one embodiment, the present invention provides for culture media that include lactose and glycerol within the ranges described above. For example, the present invention provides for culture media that include about 0.01% w/v to about 3% w/v of lactose, and about 0.1% w/v to about 5% w/v of glycerol.

In one embodiment, the present invention provides for culture media that include glucose and glycerol within the ranges described above. For example, the present invention provides for culture media that include about 0.001% w/v to about 0.5% w/v of glucose and about 0.1% w/v to about 5% w/v of glycerol.

In one embodiment, the present invention provides for culture media that include glucose, lactose, and glycerol within the ranges described above. For example, the present invention provides for culture media that include about 0.001% w/v to about 0.5% w/v of glucose, about 0.01% w/v to about 3% w/v of lactose, and about 0.1% w/v to about 5% w/v of glycerol. In another embodiment, the present invention provides for culture media that include about 0.01% w/v to about 0.02% w/v of glucose, about 0.4% w/v to about 0.6% w/v of lactose, and about 0.7% w/v to about 0.9% w/v of glycerol. In yet another embodiment, the present invention provides for culture media that include about 0.015% w/v of glucose, about 0.5% w/v of lactose, and about 0.8% w/v of glycerol.

In one embodiment, the present invention provides for culture media that include glucose and dicarboxylic acid within the ranges described above. For example, the present invention provides for culture media that include about 0.001% w/v to about 0.5% w/v of glucose, and about 0.05% w/v to about 4% w/v of dicarboxylic acid.

In one embodiment, the present invention provides for culture media that include lactose and dicarboxylic acid within the ranges described above. For example, the present invention provides for culture media that include about 0.01% w/v to about 3% w/v of lactose, and about 0.05% w/v to about 4% w/v of dicarboxylic acid.

In one embodiment, the present invention provides for culture media that include glycerol and dicarboxylic acid within the ranges described above. For example, the present invention provides for culture media that include about 0.1% w/v to about 5% w/v of glycerol, and about 0.05% w/v to about 4% w/v of dicarboxylic acid.

In some embodiments, it may be possible to exclude dicarboxylic acid from the medium. When pH control of the media is desired, pH can in the alternative be controlled or buffered with the addition of other pH controlling or buffering agents known in the art, e.g., carbonates, non-carbon sources, phosphates, or other buffering substances. The control of medium pH can also be achieved using fermentation equipment with sensor probes and feedback loops to control pH by addition of acids or bases in an automated manner.

The factorial evolved medium compositions of this invention overcome the problem of different patterns of carbon source utilization, and correspondingly, lead to high correlation of heterologous protein expression in either small-scale or large-scale protein production.

The factorial evolved medium compositions of this invention overcome the deficiency of the original auto-induction medium by Studier, which did not provide for same performance of cultures grown under aerobic or anaerobic conditions. In contrast, using the media compositions of the present invention, expression of heterologous proteins can be achieved regardless of the culture oxygenation state, i.e. regardless whether the conditions are aerobic or anaerobic.

In one example, the present invention uses the previously unrecognized concept that expression of heterologous proteins in bacterial cultures is a function of the interplay between the amount and type of carbon sources in the media, the lac repressor, and the types of plasmid used for expression, the types of promoters used for protein expression, and the plasmid copy numbers.

In one embodiment, the present invention has provided an unexpected result that auto-induction is a complex interplay of the lad repressor concentration produced by the plasmid, O₂ concentration, and medium formulation. Auto-induction is much more complicated than was previously observed. Thus, in one embodiment, the present invention teaches how to manipulate the culture conditions in order to improve auto-induction.

Having lad repressor is typically desirable, but high level interferes with the auto-induction protocol, which therefore often results in auto-induction resulting in low or no expression. Not wanting to be bound by the following theory, this might be a consequence of the level of lad repressor produced by different expression vectors. One way to overcome this problem is by designing culture media according to the present invention. In some embodiments of the present invention, attenuating the lad repressor level gives a further increase in performance, i.e., enhanced expression of recombinant proteins.

The batch addition of IPTG is the most frequently used method for induction of protein expression from the lac operon. This often leads to rapid and strong induction of protein expression. Since IPTG cannot be metabolized, this induction is irreversible and thus not under control of other cellular processes. In contrast, auto-induction occurs under control of natural cellular networks that sense the energy and nutritional status of the cell. In certain embodiments of the methods described herein, protein expression may occur over a multi-hour period (Blommel et al., 2007, Biotechnol. Prog. 23: 585-598), which may permit continued growth of the host cell even as expression continues. This increases volumetric productivity of the expression process. Experimental results also suggest that auto-induction is compatible with metal incorporation (Pierce et al., 2007, Biochemistry 46: 8569-8578) and cofactor incorporation (Bailey et al., 2007, Protein Expr. Purif. 57: 9-16), and with post-translational modifications (Zornetzer et al., 2006, Protein Expr. Purif. 46: 446-455).

The methods of the present invention also help obtain information about the physiological basis for the improved performance, revealed by the factorial evolution relative to the starting conditions. The combinations of promoters and carbon sources in the bacterial growth medium can influence the pattern of carbon source utilization, and by corollary, either favorably or unfavorably modify the pattern of heterologous protein expression.

Using the factorial media evolution approach of the present invention, it is possible to determine an optimal media composition for the growth of a chosen microorganism that expresses a desired heterologous protein. An example of this is how illustrated by the possibility to determine the optimal conditions when glycerol (used for cell growth and protein expression) and lactose (used for gene expression) are consumed simultaneously. A variety of other carbon sources can be substituted. This is exemplified below for studies with rhamnose, the rhamnose promoter, and engineered Escherichia coli (E. coli) strains such as those provided by Promega (Madison, Wis.).

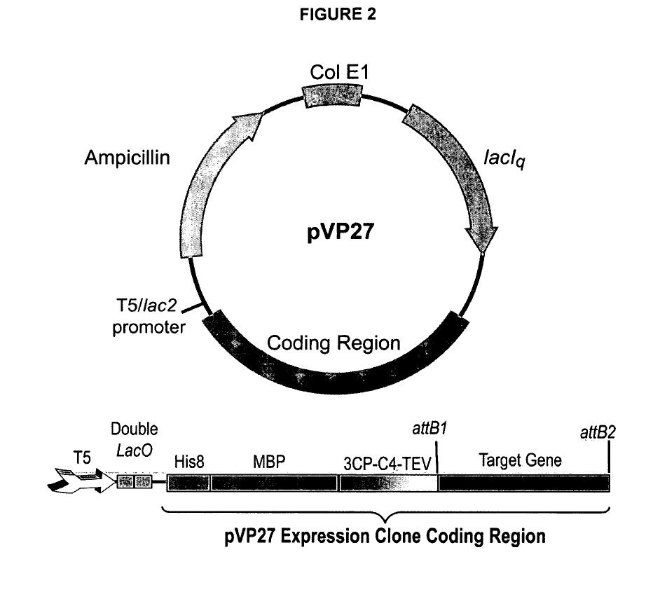

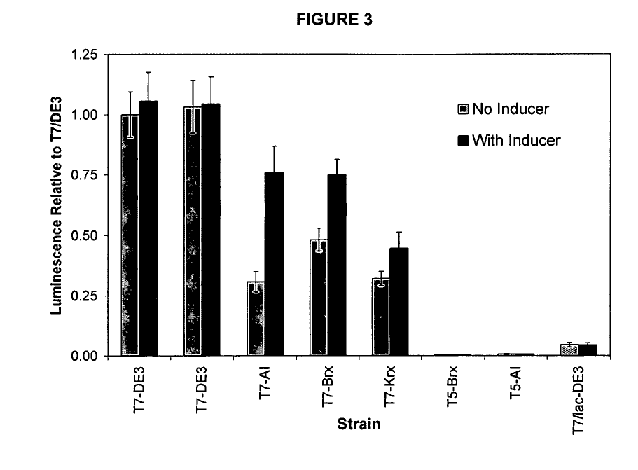

The present invention contemplates the use of a variety of expression vectors that can be recombinantly engineered to express heterologous proteins. FIG. 2 illustrates a restriction map of a T5/lac2 expression vector, an example of a vector useful for practicing the present invention. This expression vector has several desirable properties, including high level of LacI expression, low level of basal protein expression, and does not require T7 RNA polymerase. This expression vector is based on the pVP27 plasmid. However, many other expression vectors can be useful for practicing the invention, where a promoter of choice and other regulatory regions can be operably linked to a protein whose expression is desired. Preferably, the protein is heterologous.

The term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g., enhancers) in an expression cassette. In one example of the present invention, useful promotes that can be operably linked to heterologous DNA sequence that encode desired proteins include, but are not limited to, a lac promoter, a T7 promoter, a T7/lac promoter, a T5 promoter, or a T5/lac promoter.

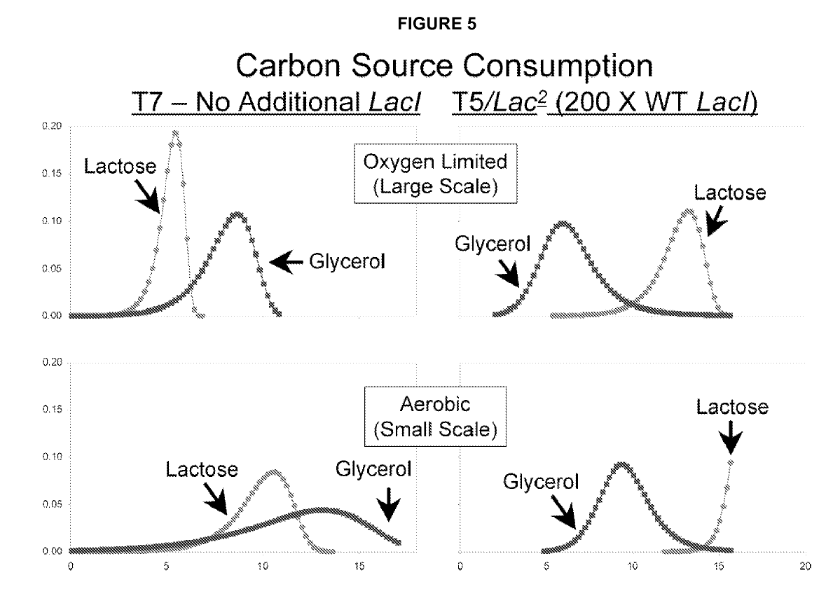

For T7 promoter systems with low levels of lac repressor (LacI) lactose is a preferential carbon source, leading to early expression in either oxygen-limited (large-scale) or aerobic (small scale) work. However, these systems have relatively low control of basal expression of gene expression, which is less desirable for process development. For T5/lac2 expression systems (Qiagen, Valencia, Calif.), the basal expression is nearly 200-fold lower than T7 systems. This property is highly desirable for heterologous expression. However, in this system lactose is not a preferred carbon source, but is utilized after all glycerol is consumed. This fact strongly switches the expression to late in the cell growth, resulting in a loss of protein expression yield. The factorial medium evolution of this invention helps to address this problem by adjusting the carbon composition of the medium so that lactose must be consumed earlier in the cell growth due to carbon limitation. For example, FIG. 5 shows carbon source consumption patterns, i.e., specific consumption of carbohydrates as a function of the cell density. Note that cell density achieved is a function of the total amount of carbon in the medium that has been consumed during the cell growth. In FIG. 5, abscissas indicate cell density measured as absorbance at 600 nm. The two left panels in FIG. 5 show the T7 promoter with no additional LacI repressor. The two right panels in FIG. 5 show the T5 promoter with 200-fold increase in LacI repressor. Lactose consumption (used for gene expression) is shown in gray filled circles; glycerol consumption (used for cell growth and protein expression) is shown in dark filled squares. In a lac promoter system, glycerol and lactose utilization is controlled by a number of physiological inputs including bacterial host catabolite repression, and surprisingly, the level of lac repressor produced by the expression plasmid. In this case, lactose consumption is strongly disfavored under all growth conditions.

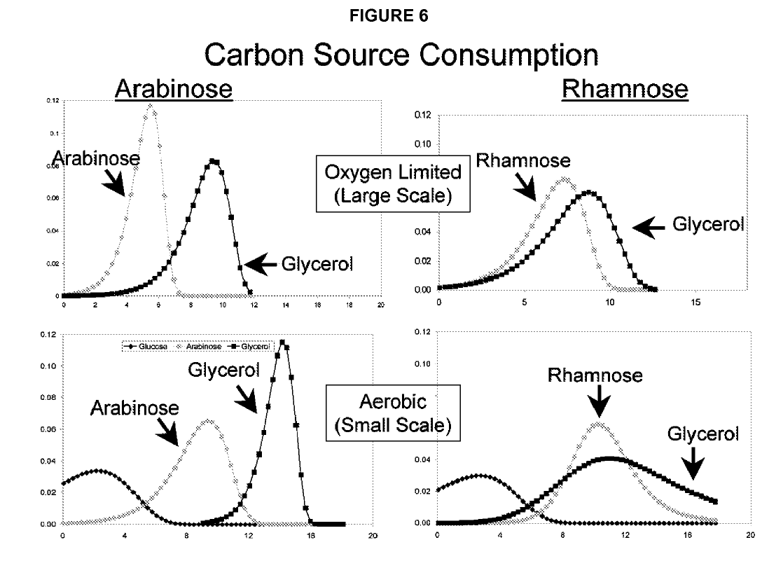

The present invention also provides for oxygenation-related considerations when designing methods and compositions for the growth of microorganisms. For example, it was discovered that small-scale expression is inherently aerobic and thus corresponds to a condition where the inducing carbon source, lactose, is the last consumed in the cycle of bacterial growth and expression. In contrast, large-scale expression is inherently oxygen-limited and thus may lead to a condition where the inducing carbon source, lactose, is consumed simultaneously with glycerol, leading to earlier expression and higher levels of expression due to the continuation of cell growth and availability of multiple carbon sources. In one aspect of the invention, the strong relationship between oxygenation state of the growth culture (small- or large-scale production) and gene expression was decoupled. This is exemplified in FIG. 6, which illustrates carbon source consumption patterns. The panels on the left show data obtained using arabinose, an often-used inducer along with the arabinose promoter (Invitrogen Corp., Carlsbad, Calif.). This combination does not provide simultaneous use of glycerol and uptake of arabinose (FIG. 6, left side). The panels on the right in FIG. 6 show data obtained using rhamnose as an inducer. In this case, consumption of glycerol and rhamnose is simultaneous, promoting strong culture growth at the same time as gene expression is induced. Thus, rhamnose (FIG. 6, right side) can be used as an inducing sugar in a properly constructed expression host to collapse the phases for consumption of glycerol and rhamnose regardless of culture oxygenation state. This leads to more predictable and more easily scalable gene expression.

The present invention also provides for carbon sources and concentration, as well as promoter systems that can be used for improved gene expression. A skilled artisan will know to substitute the frequently used glucose for alternate carbon sources. For example, carbon sources can be other monosaccharides, e.g. fructose. The use of fructose will results in less acidification; therefore, if fructose is used, then it might be possible to decrease the amount of, or even eliminate the use of, dicarboxylic acid.

According to the method of factorial evolution of the present invention, further improvements in protein production for a variety of expression promoters and a variety of bacterial expression host strains are possible. Examples of other expression promoters useful for practicing the present invention include T7, T5, arabinose, rhamnose, benzoate, and tetracycline. The utility of this invention can further be increased, for example, by expression strain engineering. As well, the utility of the invention can be increased by identification of methods to further decrease the level of basal expression from the rhamnose promoter system. Accordingly, examples of other expression host strains include minimal genome strains and engineered strains to have modified rhamnose metabolism, etc.

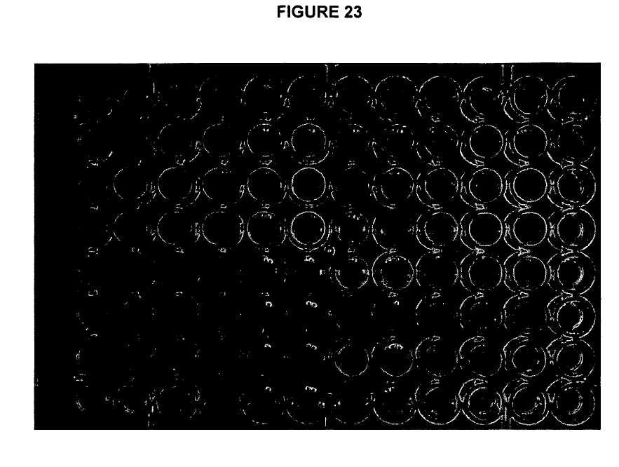

Factorial evolution of medium composition can be used to improve the correlation between results of small-scale screening of heterologous expression in E. coli host cells and large-scale protein expression in the same E. coli cells. In one exemplary embodiment, the new medium composition was used for protein production at the University of Wisconsin Center for Eukaryotic Structural Genomics. The new medium composition provides notable improvement relative to that obtained with the previous Studier medium, which is represented by wells F2 and F10 in FIG. 23. The data obtained also show an improvement in correlation between small-scale and large-scale production of proteins from ˜50% before the factorial medium was used to ˜80% after the factorial medium was used. This correlation provides an important process improvement for the protein production efforts.

In one aspect, a medium array such as the one exemplified in FIG. 23 can be used to express proteins at lower cell density and aerobic conditions when less total sugars are present or express at high cell density and microaerobic conditions when more sugars are present. The multi-well plate format described herein (e.g. see FIG. 23 and accompanying text) allows a fine-grained assessment of induction conditions for proteins of focused interest, such as intensity of induction, expression at different cell densities, etc., or investigation of induction in early-, mid-, or late-log conditions.

Using auto-induction media and methods according to the present invention, the Center for Eukaryotic Structural Genomics (CESG) at the University of Wisconsin-Madison has already expressed in Escherichia coli over 300 proteins from humans, Arabidopsis, mouse and human stem cells in the time since Apr. 9, 2007 as indicated by the National Institutes of Health public database TargetDB, and over 100 of these have been successfully purified and provided for more detailed biophysical, functional, and structural characterizations. This is a high success rate for eukaryotic proteins expressed in Escherichia coli.

The method of the present invention can be used for achieving improved levels of protein expression in a variety of prokaryotic and eukaryotic cells. In one embodiment, prokaryotic cell types useful for practicing the invention include bacteria. In an alternative embodiment, eukaryotic cell types useful for practicing the invention include yeast and mammalian cells.

### EXAMPLES

It is to be understood that this invention is not limited to the particular methodology, protocols, subjects, or reagents described, and as such may vary. It is also to be understood that the terminology used herein is for the purpose of describing particular embodiments only, and is not intended to limit the scope of the present invention, which is limited only by the claims.

The following examples are offered to illustrate, but not to limit the claimed invention.

### Chemicals

Unless otherwise stated, bacterial growth reagents, antibiotics, routine laboratory chemicals, and disposable labware were from Sigma-Aldrich (St. Louis, Mo.), Fisher (Pittsburgh Pa.), or other major distributors. L-SeMet was from Acros (Morris Plains, N.J.). Preparations of standard laboratory reagents were as described (Sambrook and Russell, 2001, Molecular Cloning: A Laboratory Manual, Cold Spring Harbor Laboratory Press, Cold Spring Harbor, N.Y., Vol. 3, pp 15.44-15.48). The 2-L polyethyleneterepthalate beverage bottles used for bacterial cell growth were from Ball Corporation (Chicago, Ill.).

### Expression Strains

The methionine auxotroph Escherichia coli B834 [genotype F⁻ ompT hsdS_{B}(r_{B}⁻m_{B}⁻) gal dcm met, as described in Wood, 1966, J. Mol. Biol. 16: 118-133] was used for expression studies with T5 promoter plasmids, while E. coli B834(DE3) [genotype F— ompT hsdS_{B}(r_{B}⁻m_{B}⁻) gal dcm met λDE3] was used for studies with T7 promoter plasmids (EMD Biosciences/Novagen, Madison, Wis.). Both expression hosts were transformed with pRARE2 (EMD Biosciences/Novagen) for rare codon adaptation. The pRARE2 plasmid conferred chloramphenicol resistance.

### Expression Vectors

In one example, Table 2 summarizes relevant properties of expression vectors evaluated in this work. pFN6K (Promega) and pET32 (EMD Biosciences/Novagen) are commercially available. The vectors pVP38K, pVP58K, pVP61K and pVP62K were created from pQE80 (Qiagen) by removal of a non-functional chloramphenicol acetyltransferase coding region and by replacement of the beta-lactamase coding region with an aminoglycoside 3′-phosphotransferase coding region conferring kanamycin resistance. pVP38K and pVP61K contain the strong lacI^{q} promoter from pQE80, 5′-GTGCAAAACCTTTCGCGGTATGGCATGAT-3′ (SEQ ID NO:1) [the point mutation responsible for the lacIq genotype is underlined], while the wild-type lacI promoter was restored by PCR in pVP58K and pVP62K, 5′-GCGCAAAACCTTTCGCGGTATGGCATGAT-3′ (SEQ ID NO:2). pVP61K and pVP62K also incorporate the gene for tobacco vein mottling virus (TVMV) protease with low-level constitutive expression so that co-transformation with a separate plasmid encoding the protease is not needed to achieve in vivo proteolysis.

TABLE 2
Expression vectors

| | Target | | | Relative | | |
|---|---|---|---|---|---|---|
| Expression | Gene | Target | Promoter | LacI | | |
| Vector^{a} | Promoter^{b} | Gene^{c} | for lacI^{d} | expression^{e} | Fusion Tag^{f} | Abbreviation^{g} |
| pFN6K | T7 | Photinus | None | 1 | N-terminal HQ | T7-Luc |
| | | luciferase | | | | |
| pET32 | T7-lacO | Photinus | lacI | 20 | N-terminal HQ | pET32-Luc or |
| | | luciferase | | | | T7-lacI-Luc |
| pVP58K | T5-lacO₁- | Photinus | lacI | 20 | N-terminal HQ | T5-lacI-Luc |
| | lacO₂ | luciferase | | | | |
| pVP38K | T5-lacO₁- | Photinus | lacI^{q} | 200 | N-terminal HQ | T5-lacI^{q}-Luc |
| | lacO₂ | luciferase | | | | |
| pVP61K | T5-lacO₁- | Enhanced | lacI^{q} | 200 | MBP-TVMV-His₈-TEV^{h} | T5-lacI^{q}-eGFP |
| | lacO₂ | GFP | | | | |
| pVP62K | T5-lacO₁- | Enhanced | lacI | 20 | MBP-TVMV-His₈-TEV^{h} | T5-lacI-eGFP |
| | lacO₂ | GFP | | | | |

^{a}pFN6K is from Promega (Madison, WI). pET32 is from Novagen (Madison, WI). Other vectors were created as part of this work.
^{b}The promoter and operator construction used for expression of the target gene. In pET32, a single copy of lacO is located 3′ to the T7 promoter. In the T5 vectors, lacO₁ is placed between the −35 and −10 regions of the ribosome binding site and lacO₂ is located between the −10 region and the start codon of the expressed gene. lacO₁ is truncated from the full length lacO₂, so may not retain the same function.
^{c}Target gene in the expression plasmid.
^{d}Promoter used for expression of lacI from the expression plasmid.
^{e}Relative level of lacI expression as compared to E. coli BL21 containing pFN6K, which includes contributions from copy number of the plasmid and relative strength of the lacI or lacI^{q} promoters.
^{f}N-terminal fusion tag on the expressed target protein.
^{g}Abbreviation for the expression plasmid used in the text.
^{h}The fusion protein is cleaved in vivo by TVMV protease to release SerHis₈GluAsnLeuTyrPheGln-AlalleAle-eGFP.

### Protein Targets

pFN6K expresses Photinus luciferase as an N-terminal fusion to (HisGln)₃ under control of the T7 promoter. Photinus luciferase was also expressed in the T7-lacI plasmid (pET32) and T5 promoter plasmids conferring both high (pVP38K, pVP61K) and medium (pVP58K, pVP62K) levels of LacI. The luciferase gene was amplified by PCR from pFN6K and the appropriate restriction sites were incorporated into the 5′ and 3′ primers. Primers were from IDT (Coralville, Iowa). The NdeI and HindIII restriction sites were used for cloning into pET32; the NcoI and HindIII restriction sites were used for cloning into pVP38K and pVP58K. The luciferase expressed from each expression vector investigated had an identical primary sequence including an N-terminal (HisGln)₃ tag.

The enhanced green fluorescent protein (eGFP) gene was assembled by overlap PCR. The eGFP gene was subsequently amplified to add the SgfI and PmeI restriction sites required for Flexi-vector cloning (Blommel et al., 2006, Protein Expr. Purif. 47: 562-570) and transferred into pVP61K and pVP62K. eGFP was initially expressed from these vectors with an N-terminal maltose binding protein fusion that underwent in vivo proteolysis by tobacco vein mottling virus (TVMV) protease to liberate SerHis₈AlaSerGluAsnLeuTyrPheGInAlaIleAla-eGFP (SEQ ID NO:3-eGFP).

### Media Formulations

The non-inducing media and the auto-induction media are derived from earlier reports on the development and use of auto-induction (Studier, 2005, Protein Expr. Purif. 41: 207-234; Tyler et al., 2005, Protein Expr. Purif. 40: 268-278; Sreenath et al., 2005, Protein Expr. Purif. 40: 256-267). All media contained 34 μg/mL of chloramphenicol and either 100 μg/mL of ampicillin or 50 μg/mL of kanamycin, depending on the selectable marker of the expression plasmid.

A 50× amino acids solution (1 L) was prepared from 10 g each of sodium glutamate, lysine-HCl, arginine-HCl, histidine-HCl, free aspartic acid, and zwitterionic forms of alanine, proline, glycine, threonine, serine, glutamine, asparagine, valine, leucine, isoleucine, phenylalanine and tryptophan.

A 5000× trace metals solution (100 mL) was prepared from 50 mL of 0.1 M FeCl₃.6H₂O dissolved in ˜0.1 M HCl, 2 mL of 1 M CaCl₂, 1 mL of 1 M MnCl₂.4H₂O, 1 mL of 1 M ZnSO₄.7H₂O, 1 mL of 0.2 M COCl₂.6H₂O, 2 mL of 0.1 M CuCl₂.2H₂O, 1 mL of 0.2 M NiCl₂.6H₂O, 2 mL of 0.1 M Na₂MoO₄.5H₂O, 2 mL of 0.1 M Na₂SeO₃.5H₂O and 2 mL of 0.1 M H₃BO₃ and 36 mL of deionized water.

A 1000× vitamins solution (100 mL) for the non-inducing medium was prepared from 2 mL of 10 mM nicotinic acid, 2 mL of 10 mM pyridoxine-HCl, 2 mL of 10 mM thiamine-HCL, 2 mL of 10 mM p-aminobenzoic acid, 2 ml of 10 mM pantothenate, 5 mL of 100 μM folic acid, 5 mL of 100 μM riboflavin, 4 mL of 5 mM vitamin B₁₂ solution and 76 mL of sterile water. A 1000× vitamins solution (100 mL) for the auto-induction medium was the same as above except that the volume of the vitamin B₁₂ solution was replaced with sterile water.

A 20× source of nitrogen, sulfate, and phosphorous (1 L), was prepared using 68 g of KH₂PO₄, 71 g of Na₂HPO₄, 53.6 g of NH₄Cl, and 14.2 g of Na₂SO₄ dissolved in sterile water.

A non-inducing medium for starting inocula (1 L) was prepared using 50 mL of 20× nitrogen, sulfate, and phosphorous mix, 0.5 g of MgSO₄, 20 mL of the 50× amino acids solution, 0.2 mL of the 5000× trace metals solution, 1 mL of the 1000× vitamins solution for the non-inducing medium, appropriate antibiotics, and 0.8% (w/v) glucose with the balance sterile water.

The auto-induction medium contained the ingredients listed above for the non-inducing medium with the noted omission of B₁₂ from the 1000× vitamins solution (Sreenath et al., 2005, Protein Express. Purif 40: 256-267) and changes in the amino acids and carbon sources as described next. For expression of unlabeled proteins, the medium contained 0.2 mg/mL of methionine. For expression of selenomethionine labeled proteins, the medium contained 0.01 mg/mL of methionine and 0.125 mg/mL of selenomethionine. The concentrations of the carbon sources (glucose, glycerol, lactose) in the auto-induction medium were varied as part of a five level, three-parameter factorial design in the following range of carbohydrate concentrations (w/v): glucose, 0 to 0.1%; glycerol 0 to 1.2% and lactose from 0 to 0.6%. Succinate was maintained at 0.375% for all media formulations.

The design points were based on two full three level cubic factorials, with one nested within the other (Myers and Montgomery, 2002, Response surface methodology: process and product optimization using designed experiments, 2nd ed., Wiley, New York). This gave a total of 53 independent medium compositions (the inner and outer factorial shared a common center point). In this design, the center points were replicated four times and the face-centered points along the lactose and glycerol axes were duplicated. These conditions were conveniently arranged into an 8×8 array within a 96-well growth block.

Variations of the media containing either methionine alone or selenomethionine and methionine were tested separately. The composition of the media used for selenomethionine-labeling was tested in a factorial design space comprised of the inner factorial (32 data points per experiment including replicates) except in the case of work with the pET32 expression vector where the full nested factorial was tested. This combination gave a total of 512 expression experiments.

### Protein Expression

Starting inocula were grown to saturation overnight in the non-inducing medium using either 96-well growth blocks having a capacity of 2 mL per well (Qiagen) or in Erlenmeyer flasks. For the growth blocks, 400 μL of the medium was used per well. For the Erlenmeyer flasks, the volume of starting inoculum was less than 10% of the total flask volume in order to promote aerobic growth. All culture growth was done at 25° C. using either plate or platform shakers.

Small-scale expression trials were carried out in 96-well growth blocks. A 20-μL aliquot of the starting inoculum was transferred to 400 μL of the auto-induction medium and incubated for 24 h at 25° C. on a plate shaker. After the incubation period, an aliquot (100-200 μL) of each 400-μL culture was transferred into a 96-well PCR plate. These samples were directly frozen at −80° C. without a preliminary cell pelleting centrifugation step. The plates were stored at −80° C. until expression analysis. Large-scale expression was conducted in 2-L PET bottles containing 500 mL of culture medium. Samples for expression analysis were harvested and stored as for the small-scale expression trials.

### Stirred Vessel Fermentations

A Sixfors parallel six fermenter system (Infors AG, Bottmingen, Switzerland) was used to investigate the influence of aeration on the auto-induction process. Two aeration states were developed to mimic the small- and large-scale cell culture environments. For the aerobic case, which best mimics the small-scale culture in the 96-well growth blocks, airflow and agitation rate were manually adjusted to maintain dissolved O₂ above 10% of saturation. For the O₂-limited condition, which best mimics the large-scale cell culture in shaken 2-L bottles, a fixed 12 volumes of air/h was added with low agitation. Samples were taken periodically to determine cell density, protein expression, and concentration of carbon sources remaining in the growth medium. The temperature was maintained at 25° C. and the pH was passively monitored during these experiments.

### Carbon Source Analysis

An HPLC method was developed to measure the concentration of sugars and organic acids present in the expression medium. A 1-mL aliquot of the culture medium was centrifuged at 16,000 g for 3 min to pellet the cells. A 900-μL aliquot of the clarified medium was added to 100 μL of a saturated Al₂(SO₄)₃ solution to precipitate phosphate. This mixture was then heated to 90° C. for 5 min to inactivate any residual enzymatic activity. Samples were stable for at least 1 wk at 4° C. after this treatment. Prior to HPLC analysis, the samples were centrifuged briefly to remove aluminum phosphate precipitate. The clarified samples were analyzed using a Shimadzu 10A HPLC system (Shimadzu, Columbia, Md.) with RID10A refractive index detector and Coregel 87H3 organic acid analysis column (Transgenomic, San Jose, Calif.). A 20-μL sample loop was used. An isocratic 0.08 N sulfuric acid mobile phase was used for elution. The elution times of the sugars, organic acids and phosphate were determined using the known compounds as standards.

### Protein Expression Analysis

For analysis of protein expression, the PCR plates of frozen cell cultures were thawed and mixed with lysis buffer to obtain a final sample composition of 20 mM Tris-HCl, pH 7.5, 20 mM NaCl, 3 kU/mL of lysozyme (EMD Biosciences/Novagen), 0.7 U/mL of benzonase (EMD Biosciences/Novagen), 0.3 mM triscarboxyethylphosphine and 1 mM MgSO₄. The presence of culture media due to the lack of a centrifugation step prior to cell lysis did not interfere with the biological assays, SDS-PAGE, or capillary electrophoresis analysis. The samples were sonicated for 6-10 min on a plate sonicator (Misonix, Farmington, N.Y.). Samples for total protein expression were prepared for analysis by LabChip90 capillary electrophoresis (Caliper Life Sciences, Hopkinton, Mass.) as recommended by the manufacturer and were prepared for SDS-PAGE analysis as previously reported (Sreenath et al., 2005, Protein Express. Purif. 40: 256-267). The soluble protein fraction used for the biological assays and LabChip90 analysis was obtained by centrifuging the sample plates for 30 min at 2200 g. Expressed protein levels were determined by LabChip90 analysis (both eGFP and luciferase) and fluorescence (eGFP only).

### Protein and Enzyme Assays

Assays for eGFP and luciferase were performed after dilution of the soluble lysate samples with buffer containing 10 mM Tris-HCl, pH 7.5, 20 mM NaCl, and 0.1 mg/mL of acetylated bovine serum albumin (Promega). For eGFP, a 5-μL aliquot of the lysate sample was mixed with 75 μL of dilution buffer prior to measurement in the wells of a black Greiner 384 well plate (ISC Bioexpress, Kaysville, Utah). Fluorescence measurements were conducted in duplicate using a Tecan Ultra 384 plate reader (Tecan Group LTD, Männedorf, Switzerland) with 485 nm (25 nm bandpass) excitation and 525 nm (20 nm bandpass) emission filters. Luciferase luminescence assays were performed using the Bright Glo luciferase assay system (Promega) after appropriate dilution of samples to bring the luciferase concentration into the linear assay measurement range. A serial dilution of purified recombinant luciferase (Promega) was assayed as a standard on every plate. Measurements were performed in duplicate with 80 μL total volume in black Greiner 384 well plates using the Tecan plate reader in luminescence mode.

### Numerical Analysis

Carbon source consumption patterns were analyzed using Microsoft Excel and the XLFit3 curve fitting add-in (ver. 3, ID Business Solutions Ltd., Guildford, UK). The changes in sugar and organic acid concentrations with respect to time and cell density were fitted to sigmoidal functions. The apparent carbon source consumption rate was determined by taking the first derivative of the sigmoidal curve fits. Results of factorial design experiments were analyzed with SAS version 9.1 (SAS Institute, Inc., Cary, N.C.). Where expression data was available for both eGFP and luciferase, the luciferase expression level was empirically found on average to be 1.58-fold higher than the eGFP expression level based on LabChip 90 quantitation of electropherograms. For model fitting purposes, the luciferase and eGFP expression data were merged into a single data set by normalizing the luciferase expression data to the eGFP expression data. This increased the number of observations available for model fitting. Expression levels were fit to either a first order model with two factor interactions (equation 1) or a second order model without factor interactions (equation 2),

EL=[Glycerol]×RF_{Glycerol}+[Lactose]×RF_{Lactose}+[Glucose]×RF_{Glucose}+[Glycerol]×[Lactose]×RF_{GlyLac}+[Lactose]×[Glucose]×RF_{LacGlu}+[Glycerol]×[Glucose]×RF_{GlyGlu}+C (eq 1)

EL=[Glycerol]×RF_{Glycerol}+[Lactose]×RF_{Lactose}+[Glucose]×RF_{Glucose}+[Glycerol]²×RF_{Gly}²+[Lactose]²×RF_{Lac}²+[Glucose]²×RF_{Glu}²+C (eq 2)

where sugar concentrations are expressed in % (w/v), EL is the expression level, RFₙ are the fitted response factors for the different media constituents and C is a fitting constant.

Both models contained seven fitted parameters and the model with the higher R² value was chosen for each data set. Data fits were significantly improved in some cases by excluding data at zero lactose concentration due to highly non-linear expression responses observed at low lactose concentrations. To simplify the graphical representation of the response surfaces, the effect of glucose was removed before generation of response surface plots by subtracting the fitted model estimate of the glucose contribution from the response at each data point. Response surface plots were generated using MathCAD version 13.0 (Mathsoft Engineering and Education, Inc.).

### Expression in Growth Blocks and 2-L Bottles

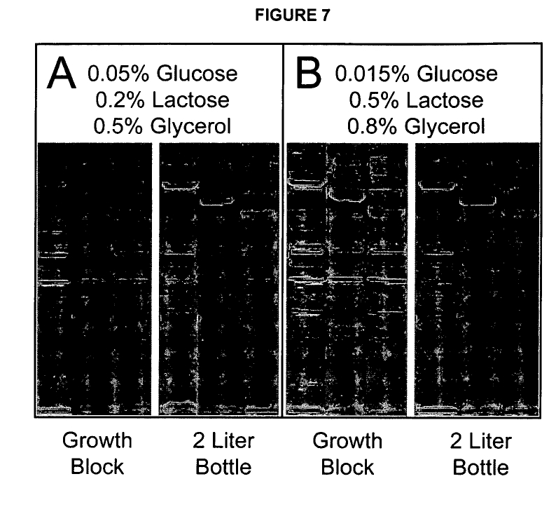

Initial experiments with auto-induction media (Studier, 2005, Protein Expr. Purif. 41: 207-234; Tyler et al., 2005, Protein Expr. Purif. 40: 268-278; Sreenath et al., 2005, Protein Expr. Purif. 40: 256-267) and T5-lacI^{q} expression plasmids revealed substantial differences between small-scale expression trials run in 96-well blocks and large-scale expression trials run in 2-L bottles. FIG. 7A shows three representative examples, which were typically characterized by low total expression in the small scale and more robust expression in the large scale. Surprisingly, higher cell densities were often obtained from the small-scale trials, which suggested more efficient use of the total carbon sources added. This poor correlation limited the predictive utility of the small-scale trials.

FIG. 7 shows images of SDS-PAGE demonstration of scale dependence during auto-induction. Total cell lysates are shown for three structural genomics target proteins (from left to right At3g17820, At1g65020, and BC058837) expressed as MBP fusions from a T5-lacI^{q} expression vector. FIG. 7A, expression in the original auto-induction medium formulation (Studier, 2005, Protein Expr. Purif. 41: 207-234). The level of expression in growth blocks was typically much lower than obtained in 2-L bottles. FIG. 7B, expression of the same targets in a provisionally revised auto-induction medium. With the indicated modifications in carbon sources, the correlation between growth blocks (small-scale) and 2-L bottles (large-scale) was improved. This figure was assembled from pictures of different gels. No modifications were made to the images other than cutting, pasting, and resizing using Adobe Photoshop.

The initial assumption was that the large-scale trials had better aeration (Millard et al., 2003, Protein Expr. Purif. 29: 311-320) than the small-scale and that O₂-limitation led to lower protein expression in the smaller cultures. However, by comparing growth rates, pH profiles, and acetate production from the two growth methods, it became apparent that the opposite was true. In one representative experiment, the small-scale cultures reached saturation at OD₆₀₀ of 22, did not produce acetate, and maintained a stable or increasing pH while cultures grown in 2-L bottles attained an OD₆₀₀ of 8, produced significant amounts of acetate, and showed a drop in pH from 6.7 to 5.0 after 24 h of incubation. By undertaking a limited investigation of the medium composition, other formulations of glucose, glycerol and lactose were found to improve the correlation between small- and large-scale expression trials. FIG. 7B shows this result for the three representative examples from FIG. 7A. Although potentially useful, this finding did not yet clarify the origin of the differences in expression behavior dependent on culture scale.

### Properties of Expression Plasmids Studied

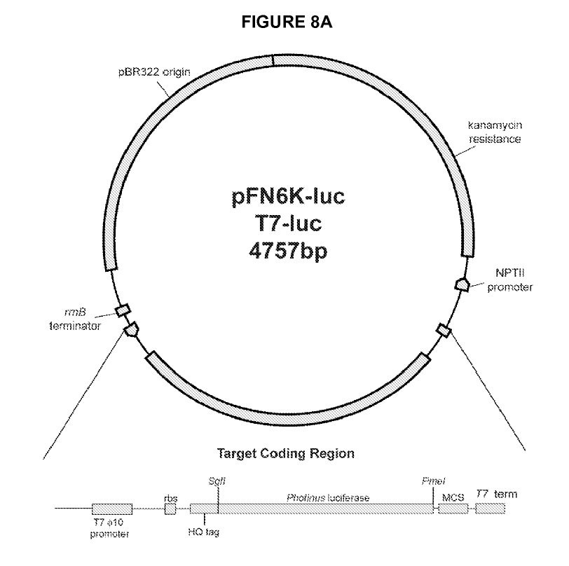

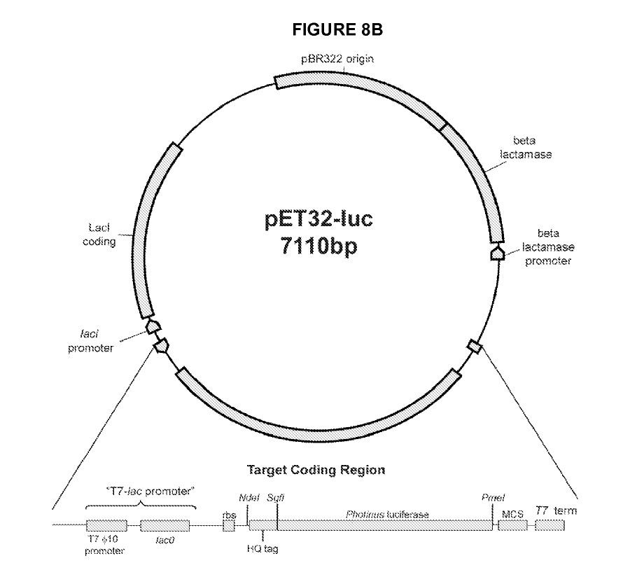

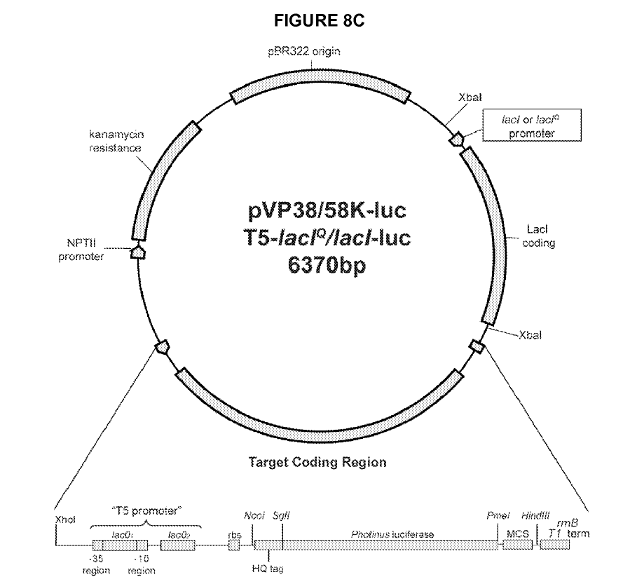

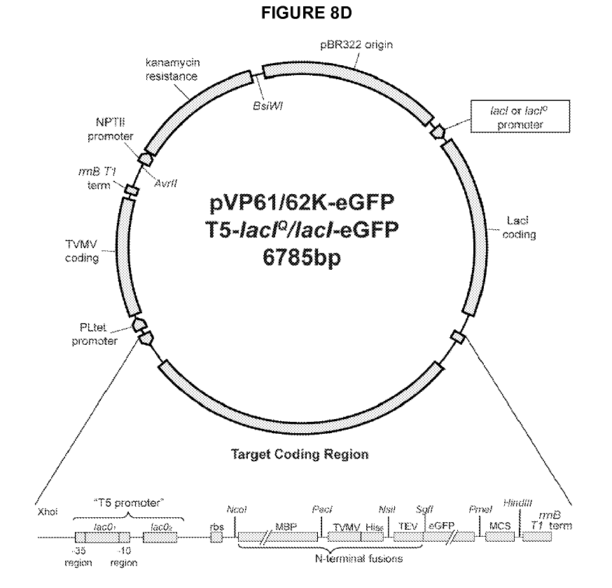

FIG. 8 illustrates maps of expression plasmids useful for practicing the invention. All four types of expression plasmids were used. Key elements of these plasmids related to the performance of auto-induction are the copy number of the plasmid, the promoter and regulator systems used to control inducible target expression and the promoter used to control constitutive expression of LacI. pFN6K has a T7 promoter, pET32 has a T7-lacO promoter, and pVP38, pVP58K, pVP61K and pVP62K have a T5-lacO₁-lacO₂ promoter. pVP38K and pVP61K have the lacI^{q} promoter controlling expression of LacI, while pVP58K and pVP62K contain the wild-type lacI promoter. Photinus luciferase was expressed from plasmids A, B, and C. Enhanced green fluorescent protein was expressed from pVP61K and pVP62K, shown in D. pVP61K and pVP62K also contain the coding region for tobacco vein mottling virus protease (TVMV) under control of the tet promoter. The expression strains used in this study do not overexpress the tet repressor, leading to low level, constitutive expression of TVMV. Due to the presence of a TVMV recognition site between the MBP and eGFP, the fusion protein is cleaved in vivo to liberate His₇-eGFP.

These expression plasmids contain the pBR322 origin of replication and have similar copy numbers of ˜15 to 20 per cell. Since only ˜10 molecules of LacI are present in wild-type E. coli, strategies have been developed to control basal expression from lac operator- repressed expression systems. pFN6K provides a T7 promoter for control of expression and no contributions from lacO or recombinant LacI to control basal expression. In contrast, pET32 provides a T7 promoter with an associated lacO sequence and constitutive expression of LacI from the plasmid. In this case, the copy number of the plasmid and the wild-type lacI promoter serve to supplement the level of LacI expression. Both pFN6K and pET32 plasmids require a lysogenic host containing T7 RNA polymerase under inducible control of the lacUV5 promoter such as E. coli B834(DE3) used here.

The pVP vectors used in this work have the T5 phage promoter (34-36) under control of two copies of the lac operator (lacO₁ and lacO₂ in FIG. 8). The lacO₁ sequence was truncated during the original construction of the pQE series of vectors, so is distinct from lacO₂, which retains the natural sequence. E. coli RNA polymerase recognizes the T5 promoter so many different E. coli expression strains can be used with this vector. pVP38K and pVP61K contain the strong lacI^{q} promoter for overexpression of LacI (originally present in pQE80), while pVP58K and pVP62K were mutated as part of this work to restore the wild type lad promoter in order to attenuate expression of LacI.

### Factorial Design of Medium Composition

Since the results of FIG. 7 showed that increasing the amounts of glycerol, lactose, and succinate—and decreasing the amount of glucose—could improve the correlation between small- and large-scale expression with the T5-lacI^{q} expression system, a factorial design approach was applied to individually optimize the media for small-scale expression using the T5-lacI, T5-lacI^{q} and pET32 (T7-lacI) plasmids. For this optimization, all media constituents were fixed except for glucose, glycerol and lactose, and these were independently varied in a factorial design approach (Swalley et al., 2006, Anal. Biochem. 351: 122-127; Myers and Montgomery, 2002, Response surface methodology: process and product optimization using designed experiments, 2^{nd} ed., Wiley, New York).

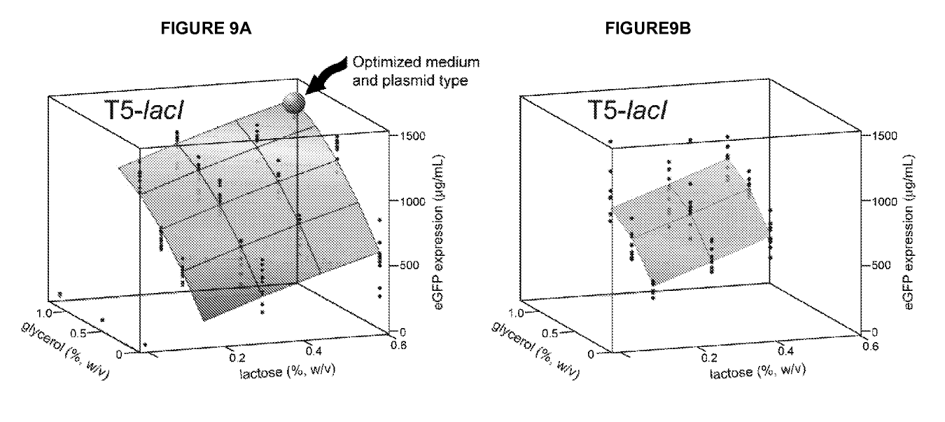

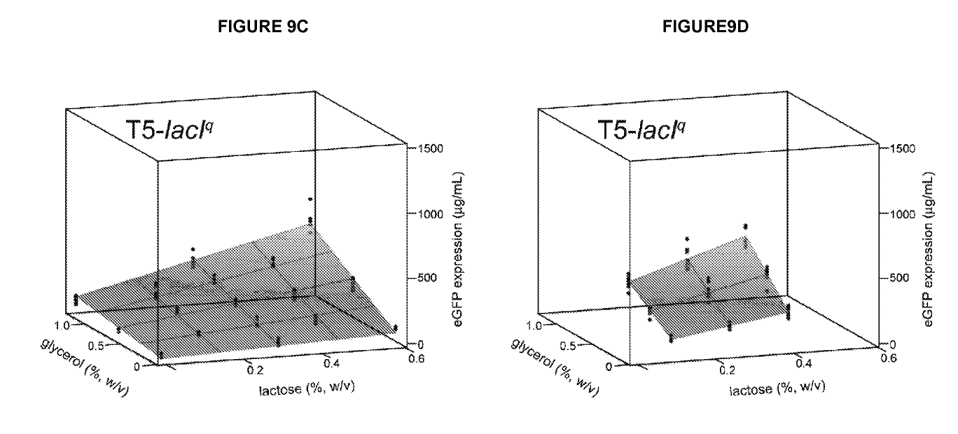

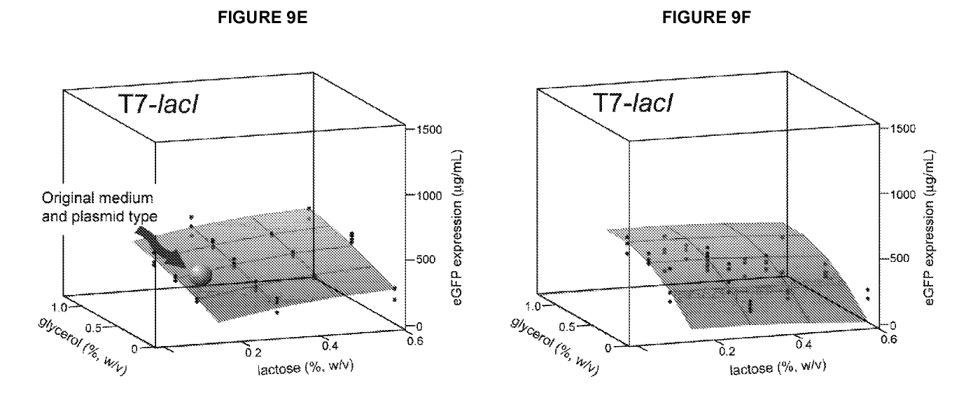

FIG. 9 shows graphs of response surfaces arising from factorial design changes in the composition of auto-induction medium and changes in LacI dosing, for expression using the T5-lacI-eGFP expression plasmid. FIGS. 9A and B, expression from T5-lacI plasmids in media containing methionine (A) or selenomethionine (B). FIGS. 9C and D, expression from T5-lacI^{q} plasmids in media containing methionine (C) or selenomethionine (D). FIGS. 9E and F, expression from T7-lacI (pET32) plasmids in media containing methionine (E) or selenomethionine (F). The response models were not extended to zero lactose concentration due to highly non-linear response with this medium composition. Response surface models are thus shown for expression results obtained in media containing methionine only (left side, including evaluation of 53 independent medium compositions) or selenomethionine (right side, including evaluation of 32 independent medium compositions for T5-lacI and T5-lac/1 or 53 compositions for pET32). The left response surface shows that variations of the carbon sources in a methionine medium can give a nearly 15-fold increase in soluble eGFP production based on the measured fluorescence, which corresponds to a range from ˜100 μg/mL of eGFP in the poorest performing composition to ˜1500 μg/mL of eGFP in the best performing composition. eGFP was used as an expression target for total soluble protein expression due to the ease of quantification through intrinsic fluorescence. Since eGFP requires O₂ for fluorophore formation, only small-scale expression experiments where O₂ was not limited were undertaken. The right side of FIG. 9A shows the response surface for the same expression experiment in media containing selenomethionine. Overall, the response surfaces for T5-lacI-eGFP expression in the methionine and selenomethionine media tracked each other closely. Indeed, among the lesser number of compositions investigated for the selenomethionine medium, soluble eGFP expression was observed in excess of 1000 μg/mL (total recombinant protein expression exceeded 2000 μg/mL if MBP expression was also accounted for).

FIG. 9B shows the response surfaces for expression from T5-lacI^{q}-eGFP. This expression system gave lower total expression than T5-lacI-eGFP, with expression levels ranging from near zero at low lactose to ˜600 μg/mL when glycerol and lactose were maximized. FIG. 9C shows the response surfaces for expression from pET32.

Table 3 shows the statistical factors for the model analysis of these two different media optimizations with the T5-lacI-eGFP expression vector. In both the methionine and selenomethionine media, a change in the glycerol concentration was most strongly correlated to a positive expression response, accounting for an estimated 38% or 36% of the modeled effect, respectively. In the methionine medium, increasing lactose concentration was also correlated with the expression response, accounting for 21% of the modeled effect. In the selenomethionine media, increasing lactose had less influence on the expression response, accounting for 13% of the modeled effect, while other higher order terms had a larger influence.

TABLE 3
Response surface effect estimates for auto-induction of eGFP
expression from the T5-lacI expression plasmid pVP62K

| METHIONINE MEDIUM | | |
|---|---|---|
| Model variable^{a} | Scaled effect estimate^{b} | p-value^{c} |
| Glucose | −0.15 | <0.001 |
| Glycerol | 0.38 | <0.001 |
| Lactose | 0.21 | <0.001 |
| Glucose² | 0.1 | 0.003 |
| Glycerol² | −0.14 | <0.001 |
| Lactose² | −0.03 | 0.54 |
| Model R² | | 0.86 |
| SELENOMETHIONINE | | |
| MEDIUM | | |
| Model variable | Scaled effect estimate | p-value |
| Glucose | 0.13 | 0.17 |
| Glycerol | 0.36 | <0.001 |
| Lactose | 0.11 | 0.24 |
| Glucose² | −0.18 | 0.049 |
| Glycerol² | −0.21 | 0.026 |
| Lactose² | 0.01 | 0.9 |
| Model R² | | 0.73 |

^{a}Variables from equation 2 used for response surface modeling based on concentrations of glucose, glycerol, and lactose, and measured expression results.
^{b}The estimated fractional contribution to the observed change in expression, with both positive and negative effects indicated.
^{c}p-values indicate the likelihood that the calculated fractional contribution contributes to the observed change;
R² value represents the overall predictive value of the models.

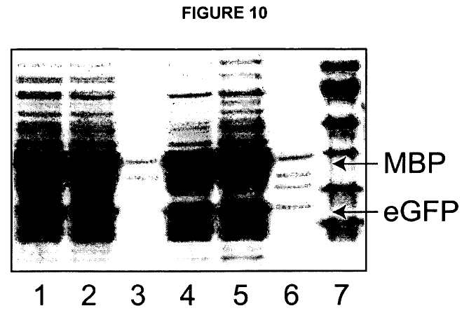

FIG. 10 shows an image of SDS-PAGE analysis of eGFP expression from T5-lacI-eGFP. In this case, stoichiometric proteolysis of the original fusion protein (70 kDa) to MBP (42 kDa) and the tagged-eGFP (29 kDa) was obtained from the constitutively expressed TVMV protease. Lanes 1, 2 and 3 show total cell lysate, soluble fraction and insoluble fraction obtained from expression in a methionine auto-induction medium containing 0.025% (w/v) glucose, 0.9% (w/v) glycerol, and 0.45% (w/v) lactose. Lanes 4, 5 and 6 show total cell lysate, soluble fraction and insoluble fraction obtained from expression in selenomethionine auto-induction medium with the same carbon source composition.

### Basal Expression Studies Using Luciferase

Luciferase was used as an expression target due to the large linear range of the luminescence assay (5-6 orders of magnitude) and a low detection limit that was useful for quantifying basal expression. Table 4 compares the basal expression of luciferase from three of the plasmid types. The unregulated T7-Luc plasmid (pFN6K) gave the highest level of basal expression in non-inducing medium and a small increase in basal expression in auto-induction medium. This result arose through expression of T7 RNA polymerase from the poorly repressed genomic lacUV5 promoter and subsequent transcription from the plasmid T7 promoter upstream of the luciferase gene. In contrast, the highly regulated T5-lacI^{q}-Luc plasmid (pVP38K) gave the lowest level of basal expression, around 1% of that from the T7-Luc plasmid, and no difference in basal expression was observed in either non-inducing or auto-induction media. The presence of two copies of lacO in the promoter region and overexpression of LacI from the plasmid contribute to this result. The T7-lacI plasmid pET32-Luc gave a 20-fold reduction in basal luciferase expression as compared to the T7 vector, but this level was still 5× higher than that observed with the T5-lacI^{q} plasmids. Results from the T5-lacI-Luc plasmid (pVP58K-Luc) suggested an expression level in the non-inducing medium similar to pET32-Luc. Thus the higher basal expression observed for the T7-lacI and T5-lacI plasmids compared to T5-lacI^{q} is likely a result of a decrease in cellular LacI and corresponding lower occupancy of the promoter lacO sites. Overall, the presence of lactose in the medium did not significantly increase basal expression of luciferase, indicating that the effects of catabolite repression and inducer exclusion are sufficiently strong to prevent premature induction of the lac operon.

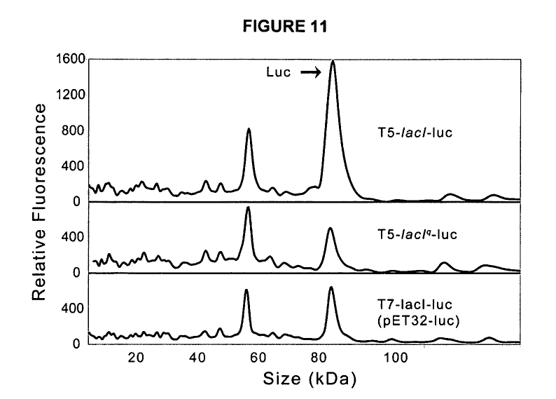

When expressed at low levels, luciferase was found to be entirely soluble. However, as expression increased beyond 100 μg/mL of culture, an increasing fraction of the luciferase was insoluble. For this reason, total luciferase expression was determined using capillary electrophoresis. FIG. 11 shows capillary electrophoresis elution profiles for luciferase expression in various medium compositions. These are graphs of LabChip90 protein electropherograms showing luciferase expression from the indicated luciferase expression plasmids. Reported luciferase expression levels were 1820 mg/L (T5-lacI, top), 500 mg/L (T5-lacI^{q}, middle), and 640 mg/L (T7-lacI, bottom). Each protein expression was obtained from methionine auto-induction medium containing 0.025% (w/v) glucose, 0.45% (w/v) lactose and 0.9% (w/v) glycerol.

TABLE 4
Basal expression of luciferase from different expression plasmids in
auto-induction media

| Expression | −Lactose^{a} | +Lactose^{b} |
|---|---|---|
| Vector | μg/mL | μg/mL |
| T7-Luc | 2.7 ± 0.3 | 2.9 ± 0.4 |
| T7-lacI-Luc (pET32-Luc) | 0.19 ± 0.04 | 0.19 ± 0.04 |
| T5-lacI^{q}-Luc | 0.03 ± 0.008 | 0.03 ± 0.004 |

^{a}Luciferase activity interpolated at a cell density of 2 (600 nm) based on measurements taken at lower and higher cell densities during exponential growth in a non-inducing medium containing 0.8% (w/v) glucose.
^{b}Luciferase activity interpolated at a cell density of 2 (600 nm) based on measurements taken at lower and higher cell densities during exponential growth in auto-inducing medium containing 0.8% (w/v) glucose and 0.1% (w/v) lactose.

### Fermentation Approach

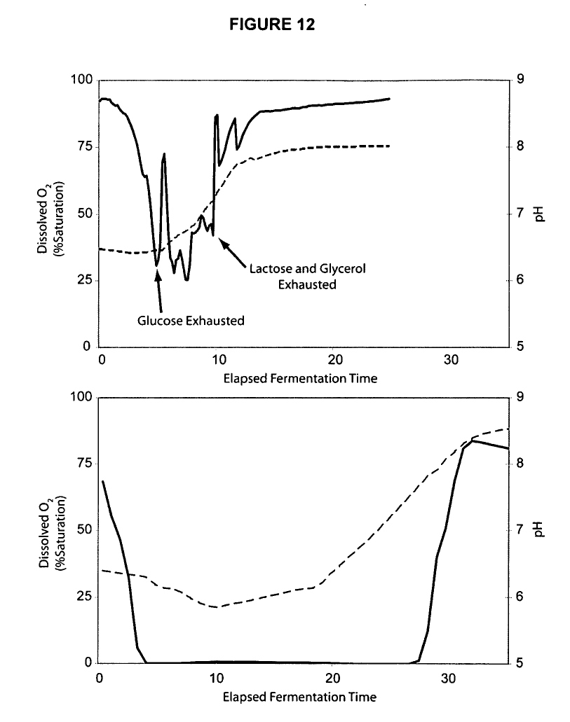

An instrument-controlled fermenter was used to investigate the correlation between carbon source utilization, O₂ saturation of the culture, and protein expression. In the fermenter, an aerobic growth condition was maintained during auto-induction by fixing O₂ at greater than 10% of saturation during the entire cell growth. The aerobic growth condition in the fermenter best represents growth of small-scale cultures in 96-well growth blocks. For comparison, a microaerobic growth condition was maintained by completing the growth phase under O₂-limitation. The microaerobic growth condition best represents growth of large-scale cultures in 2-L bottles. FIG. 12 shows dissolved O₂ and pH profiles for growth and auto-induction under these two conditions.

FIG. 12 shows graphs of dissolved O₂ (solid lines) and pH (dashed lines) profiles for aerobic (top panel) and O₂-limited (bottom panel) growth of E. coli B834 T7-Luc completed in a Sixfors instrumented fermenter. In both cases, the dissolved O₂ initially dropped as increasing cell density raised the metabolic O₂ demand. For the aerobic growth, the dissolved O₂ was maintained above 10% of saturation during the course of the experiment. The dissolved O₂ fluctuated in the aerobic fermentation during transitions from use of one carbon source to another and due to manual adjustments in agitation made to maintain aerobic conditions. The arrows indicate the times where glucose, lactose and glycerol were exhausted. For the O₂-limited growth, dissolved O₂ was below measurable levels for much of the experiment because the metabolic demand exceeds the amount of O₂ supplied. After 10 h for the aerobic case and ˜28 h for the O₂-limited case, most of the carbon sources were consumed and the dissolved O₂ increased rapidly as the metabolism ceased. For the aerobic growth, the pH was constant during glucose consumption and rose as succinate was consumed. In the O₂-limited case, the pH dropped initially as acetate was produced by fermentation. The trend was reversed as succinate, and eventually acetate, were consumed.

### Carbon Source Consumption Patterns

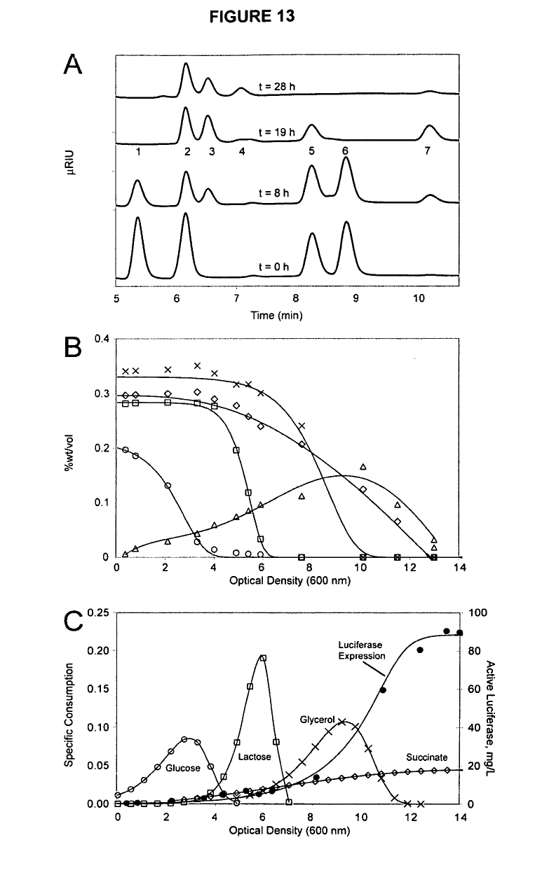

FIG. 13 shows graphs of HPLC determination of carbon source levels and carbon consumption patterns during the time course of O₂-limited auto-induction in E. coli B834 (DE3) transformed with T7-Luc. FIG. 13A: HPLC analysis of samples from different times during the fermentation. Peak identities are: 1, lactose; 2, glucose co-eluting with phosphate; 3, galactose; 4, unknown fermentation product; 5, succinate; 6, glycerol and 7, acetate. The sample from t=0 was taken immediately after inoculation of the fermenter. The middle traces show accumulation of galactose and acetate during intermediate time points and the bottom trace shows phosphate, galactose and acetate remained at the end of the fermentation. Galactose cannot be metabolized by E. coli B834 and increased as a byproduct of lactose consumption, while acetate was a byproduct of anaerobic fermentation. FIG. 13B: sigmoidal curve fitting of the relationship between change in carbon source concentration and cell density. In all cases, glucose (circles) was consumed first, and followed successively by lactose (squares), glycerol (x), succinate (diamonds) and then acetate (triangles). Acetate was initially produced and later consumed as a carbon source. FIG. 13C: first derivative of the sigmoidal curve fits, defined to be the specific consumption for each carbon source. These series have the same markers as in B. The filled circles show luciferase expression from the T7-Luc expression plasmid as determined by luminescence assay.

FIG. 13A shows representative HPLC traces obtained from the culture medium during the course of a growth of E. coli B834(DE3) with the simple T7-Luc plasmid in auto-induction medium. At t=0, lactose, glucose, succinate and glycerol are present. At t=8 h (cell density of ˜5), the glucose was entirely consumed and lactose had become the preferred carbon source, so it was being depleted from the culture medium. Acetate accumulated early in the growth and auto-induction, but was later consumed. At t=28 h (cell density of ˜13), the growth was complete and the only identified carbon sources remaining were a residual small amount of acetate and a larger amount of galactose. Galactose accumulates in the culture medium when lactose is consumed as E. coli B834(DE3) is a galactose auxotroph.

FIG. 13B shows the complete pattern of carbon source consumption during the aerobic growth of E. coli B834(DE3) transformed with T7-Luc in the auto-induction medium. In these cells, LacI is only provided by low-level constitutive expression from the bacterial genome. The carbon source concentrations were fitted as sigmoidal functions (solid lines) for illustrative purposes, and FIG. 13C shows the first derivative of these fits. In FIGS. 13B and 13C, the carbon consumption patterns are plotted relative to cell density (optical density at 600 nm), which provides a useful correlation between an easily measured experimental property and the status of the carbon sources during growth and auto-induction. For example, the transition from growth on glucose to growth on lactose occurs at a cell density of ˜5, lactose consumption is complete at a cell density of ˜7, and no consumable carbon sources are remaining when the cell density has reached ˜13. The carbon consumption pattern of E. coli BL21 lacking an expression plasmid was indistinguishable.

This pattern of carbon consumption is consistent with previous studies of E. coli diauxic growth (Inada et al., 1996, Genes Cells 1: 293-301. Thus glucose was preferentially consumed, followed by lactose, and finally glycerol. Furthermore, in these experiments, succinate was gradually consumed throughout the entire growth period and acetate was largely consumed by the end of the culture growth. In auto-induction, protein expression from the lac operon will be induced along with lactose consumption. For example, induction of T7 RNA polymerase expression under the control of a lacUV5 promoter in E. coli B834(DE3) would be expected to coincide with activation of the lac operon. Correspondingly, FIG. 13C shows that luciferase activity was detected at a cell density of ˜5 when lactose became the preferred carbon source, and continued to increase after lactose consumption was complete as glycerol and succinate were consumed.

### Effect of LacI Dosing on Carbon Consumption Patterns

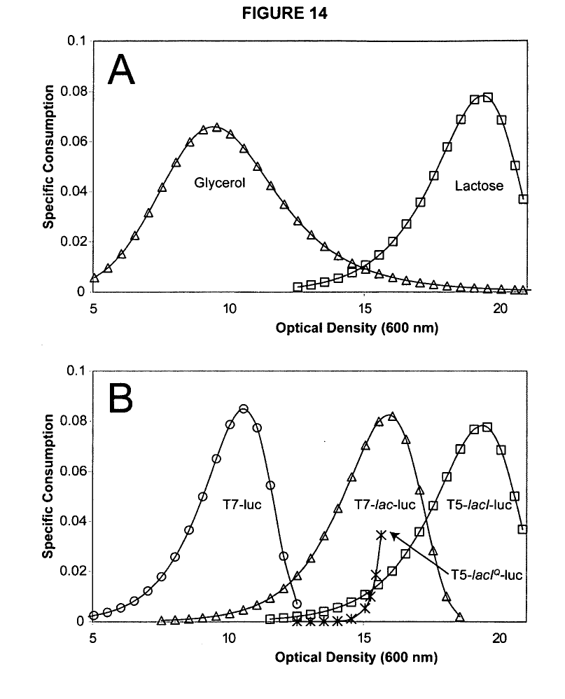

FIG. 14 shows the effect of different levels of LacI on the carbon consumption patterns during auto-induction. The consumption patterns for glycerol and lactose for E. coli B834 expressing T5-lacI-Luc (pVP58K) by aerobic auto-induction are shown in FIG. 14A. This construct provides expression of plasmid-encoded LacI from the weak lad promoter. Increasing LacI shifts the order of preference from glucose/lactose/glycerol to glucose/glycerol/lactose in aerobic culture. Glycerol is preferentially consumed before lactose in an aerobic growth with the T5-lacI expression plasmid. Thus there is a dramatic shift in the pattern of carbon consumption relative to the T7-Luc data shown in FIG. 13C, where lactose is preferentially consumed before glycerol. Consumption of glucose and succinate are not shown for clarity. FIG. 14B: specific consumption of lactose during auto-induction growth with the indicated luciferase expression plasmids. The T7-Luc expression plasmid does not supplement LacI expression. The T7-lacI-Luc and T5-lacI-Luc plasmids contain a plasmid borne copy of the lac repressor gene with a wild-type promoter and give ˜20-fold increase in the level of LacI relative to T7-Luc. The T5-lacI^{q}-Luc plasmid also contains a plasmid borne copy of the lac repressor with a lacI^{q} promoter that increases the level of LacI by ˜10-fold higher than from T7-lacI-Luc and T5-lacI-Luc. With this latter plasmid, only a small amount of lactose was consumed and culture growth was halted at a cell density of 16 (OD₆₀₀ units). In contrast, the other cultures were able to fully consume the lactose and achieved a cell density of ˜21.

T7-Luc, which provides no recombinant LacI, maximally consumed lactose at a cell density of ˜10. In contrast, pET32-Luc (a T7-lacI plasmid with constitutive plasmid-encoded expression of LacI) shifted the maximal consumption of lactose to a cell density of ˜15, while pVP58K-Luc (a T5-lacI plasmid also providing constitutive plasmid-encoded expression of LacI) behaved in a similar manner and shifted the maximal consumption of lactose to a cell density of ˜18. Finally, with pVP38K-Luc (a T5-lacI^{q}-Luc plasmid giving overexpression of plasmid-encoded LacI from the strong lacI^{q} promoter), the shift in carbon consumption pattern was so extreme that culture growth stopped in aerobic conditions before lactose could be substantially consumed (FIG. 14B, x symbols).

### Consequences of O₂ Availability During Auto-Induction

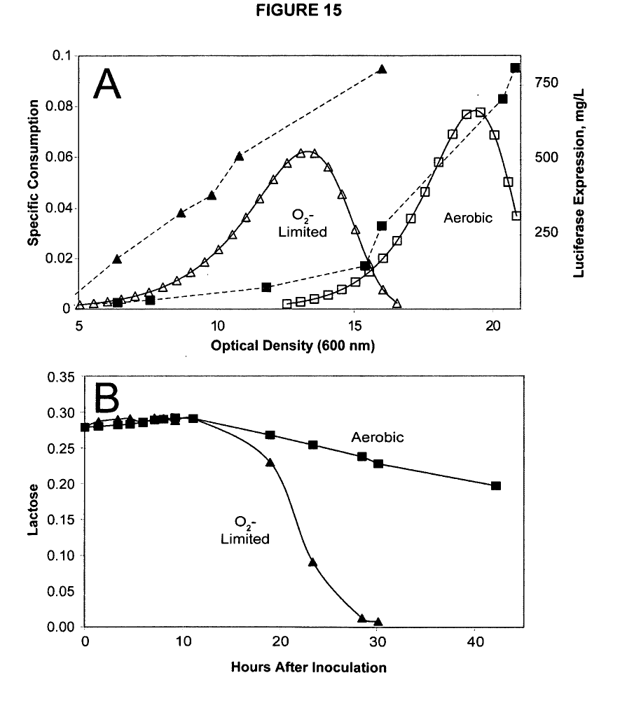

FIG. 15 shows graphs of the effect of aeration on lactose consumption with the T5-lacI-Luc expression plasmid. FIG. 15A, lactose consumption (open triangles) and protein expression (filled triangles) occurred at an earlier stage of growth in O₂-limited cultures as compared to the aerobic cultures (lactose consumption and expression measurements represented with either open or filled squares, respectively). FIG. 15B, effect of the T5-lacIq expression plasmid on lactose consumption. In O₂-limited cultures, all lactose was consumed by 30 h after inoculation. In the aerobic cultures, the cell density stopped increasing at 20 h and lactose was only slowly consumed thereafter.

FIG. 15A shows the consequences of aerobic or O₂-limited growth on the lactose consumption pattern for T5-lacI-Luc expression in E. coli B834. During aerobic growth, the maximal lactose consumption occurred at a cell density of ˜18, as shown in FIG. 14. The appearance of luciferase activity closely tracked this maximal consumption pattern, which is consistent with the relatively strong control of basal expression given by aerobic growth and the presence of recombinant LacI. For comparison, FIG. 15A also shows that O₂-limited growth during auto-induction shifted the maximal lactose consumption to a lower cell density. Thus changes in oxygenation state of the medium dramatically affected the preference for lactose consumption relative to other carbon sources. Furthermore, in the O₂-limited growth, the appearance of luciferase activity no longer closely tracked the lactose consumption pattern, but was shifted to earlier in the overall growth period. These results are consistent with a weakening of catabolite repression and consequent increase in basal expression from both the genomic lac operon (generating allolactose) and from the recombinant expression system (generating luciferase).

FIG. 15B emphasizes the strong influence of oxygenation state on the consumption of lactose with the E. coli T5-lacI^{q} expression vector. Under aerobic auto-induction conditions, lactose utilization was only weakly initiated and ˜70% of the initial lactose remained after ˜40 h. After the time when glucose, glycerol, and succinate were consumed (˜15 h), little additional cell growth or protein expression were observed. For comparison, O₂-limited auto-induction gave complete utilization of lactose between 10 and 30 h. During this time, continued cell growth and protein expression were obtained.

### Examples of Media Useful for Practicing the Present Invention

In this example, media used to vary the sugar concentrations of the auto-induction medium according to the factorial evolution process are prepared from the follow stock solutions.

A 1 L aliquot of sugar-free, methionine-containing auto-induction medium is prepared by adding by adding to 900 mL of deionized water and thoroughly mixing (in the order given) 1 mL of MgSO₄ solution, 0.2 mL of the 5000× trace metals solution, 1 mL of the 1000× non-inducing medium vitamins solution, 1 mL of the 1000× vitamin B₁₂ solution, 25 mL of the 40× succinate solution, 50 mL of the 20× nitrogen, sulfate, and phosphorous solution, 10 mL of the 50× amino acids solution, 4 mL of the 250× methionine solution, and the appropriate antibiotics. The balance of the total volume is provided by sterile water.

A 1 L aliquot of sugar-free, selenomethionine-containing auto-induction medium is prepared by adding by adding to 900 mL of deionized water and thoroughly mixing (in the order given) 1 mL of MgSO₄ solution, 0.2 mL of the 5000× trace metals solution, 1 mL of the 1000× non-inducing medium vitamins solution, 1 mL of the 1000× vitamin B₁₂ solution, 25 mL of 40× succinate solution, 50 mL of the 20× nitrogen, sulfate, and phosphorous solution, 10 mL of the 50× amino acids solution, 0.4 mL of the 250× methionine solution, 5 mL of the 250× selenomethionine solution, and the appropriate antibiotics. The balance of the total volume is provided by sterile water.

Table 5 defines how the (w/v) percentages of glucose, lactose, and glycerol are arranged in one example of the growth block format. The methionine-containing auto-induction medium is arranged into an 8×8 array within a 96-well growth block, while the selenomethionine-containing auto-induction medium is arranged into an 8×4 array. In Table 5, columns 1-8 contain methionine media while columns 9-12 contain selenomethionine labeling media. As an example for assembly of a 1 mL culture, one may place 0.9 mL of the methionine-containing auto-induction medium into position A1 of the growth block, and add 20 μL of 40% (w/v) glucose solution, 11.3 μL of 40% (w/v) lactose solution, and 7.5 μL of 40% (w/v) glycerol solution. The balance of the total volume in well A1 is provided by sterile water.

TABLE 5
Concentrations of glucose (top), lactose (middle), and glycerol
(bottom) for each expression media tested are shown in the
96 well plate format used for expression testing

| | 1 | 2 | 3 | 4 | 5 | 6 | 7 | 8 | 9 | 10 | 11 | 12 |
|---|---|---|---|---|---|---|---|---|---|---|---|---|
| A | 0.08 | 0.05 | 0.03 | 0.15 | 0.85 | 0.00 | 0.05 | 0.05 | 0.28 | 0.05 | 0.03 | 0.05 |
| | 0.45 | 0.45 | 0.45 | 0.30 | 0.80 | 0.00 | 0.20 | 0.30 | 0.45 | 0.45 | 0.45 | 0.30 |
| | 0.30 | 0.35 | 0.30 | 0.20 | 0.00 | 0.00 | 0.50 | 0.80 | 0.30 | 0.30 | 0.20 | 0.60 |
| B | 0.08 | 0.05 | 0.03 | 0.10 | 0.05 | 0.00 | 0.55 | 0.05 | 0.05 | 0.05 | 0.03 | 0.05 |
| | 0.20 | 0.30 | 0.30 | 0.30 | 0.20 | 0.30 | 0.30 | 0.30 | 0.30 | 0.30 | 0.30 | 0.30 |
| | 0.90 | 0.90 | 0.90 | 1.20 | 1.20 | 1.20 | 0.60 | 0.80 | 0.50 | 0.90 | 0.80 | 0.50 |
| C | 0.08 | 0.05 | 0.03 | 0.10 | 0.05 | 0.00 | 0.03 | 0.35 | 0.08 | 0.05 | 0.03 | 0.08 |
| | 0.45 | 0.45 | 0.45 | 0.50 | 0.80 | 0.00 | 0.30 | 0.30 | 0.45 | 0.45 | 0.45 | 0.30 |
| | 0.90 | 0.95 | 0.90 | 1.20 | 1.20 | 1.20 | 0.50 | 0.80 | 0.35 | 0.80 | 0.80 | 0.80 |
| D | 0.08 | 0.05 | 0.03 | 0.10 | 0.05 | 0.00 | 0.08 | 0.70 | 0.05 | 0.05 | 0.03 | 0.08 |
| | 0.45 | 0.45 | 0.45 | 0.35 | 0.60 | 0.80 | 0.30 | 0.30 | 0.45 | 0.45 | 0.45 | 0.30 |
| | 0.90 | 0.60 | 0.80 | 0.85 | 0.50 | 0.80 | 0.00 | 0.80 | 0.30 | 0.80 | 0.00 | 0.50 |
| E | 0.08 | 0.05 | 0.03 | 0.10 | 0.05 | 0.00 | 0.05 | 0.05 | 0.08 | 0.05 | 0.03 | 0.05 |
| | 0.15 | 0.15 | 0.15 | 0.08 | 0.00 | 0.00 | 0.45 | 0.00 | 0.15 | 0.15 | 0.15 | 0.45 |
| | 0.80 | 0.65 | 0.60 | 0.80 | 0.80 | 0.60 | 0.50 | 0.80 | 0.95 | 0.05 | 0.50 | 0.00 |
| F | 0.08 | 0.05 | 0.03 | 0.70 | 0.05 | 0.00 | 0.05 | 0.05 | 0.08 | 0.05 | 0.03 | 0.05 |
| | 0.15 | 0.15 | 0.15 | 0.95 | 0.00 | 0.00 | 0.15 | 0.00 | 0.15 | 0.15 | 0.15 | 0.15 |
| | 0.20 | 0.30 | 0.30 | 0.80 | 0.50 | 0.00 | 0.00 | 0.80 | 0.30 | 0.30 | 0.30 | 0.50 |
| G | 0.08 | 0.05 | 0.03 | 0.18 | 0.05 | 0.00 | 0.05 | 0.05 | 0.08 | 0.05 | 0.03 | 0.05 |
| | 0.30 | 0.30 | 0.30 | 0.30 | 0.30 | 0.30 | 0.30 | 0.30 | 0.80 | 0.30 | 0.20 | 0.30 |
| | 0.30 | 0.30 | 0.50 | 0.90 | 0.00 | 0.00 | 0.50 | 1.20 | 0.30 | 0.30 | 0.30 | 0.90 |
| H | 0.08 | 0.05 | 0.03 | 0.10 | 0.05 | 0.00 | 0.05 | 0.05 | 0.08 | 0.05 | 0.02 | 0.05 |
| | 0.15 | 0.15 | 0.15 | 0.00 | 0.50 | 0.00 | 0.30 | 0.30 | 0.15 | 0.15 | 0.15 | 0.30 |
| | 0.20 | 0.90 | 0.90 | 1.20 | 1.20 | 1.20 | 0.30 | 0.05 | 0.90 | 0.90 | 0.90 | 0.30 |

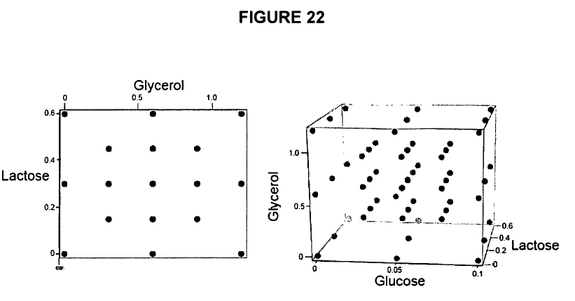

FIG. 22 shows graphs with representations of the factorial experimental design experimental space. The design points limited to glycerol and lactose are shown in FIG. 22A while FIG. 22B is a three dimensional projection of all design points from the three-factor five-level factorial.

FIG. 23 is an image of a 96 well plate containing diluted eGFP expression lysates from the media listed in Table 5 illuminated with a 340 nm light source. Note that black 384 well plates were used for quantitation, not the clear 96 well plate shown here.

### Example of an Auto-Induction Method

Auto-induction medium includes a mixture of carbon and energy sources. Glucose is the preferred source for E. coli and is utilized during the early stages of growth. Lactose and glycerol serve as carbon and energy sources during later stages of growth and recombinant protein production. Succinate (or other organic acids such as aspartate or glutamate) may be included to help maintain the culture pH and to act as additional sources of carbon and nitrogen. The consumption of these individual carbon sources by E. coli has been extensively studied and in some cases, in combination (as is the case for glucose-lactose diauxic growth). This work demonstrates the importance and possible advantages of considering the interactions between media composition, LacI expression and oxygenation state in the function of auto-induction systems for protein production in E. coli.

### Comparison of Response Surfaces

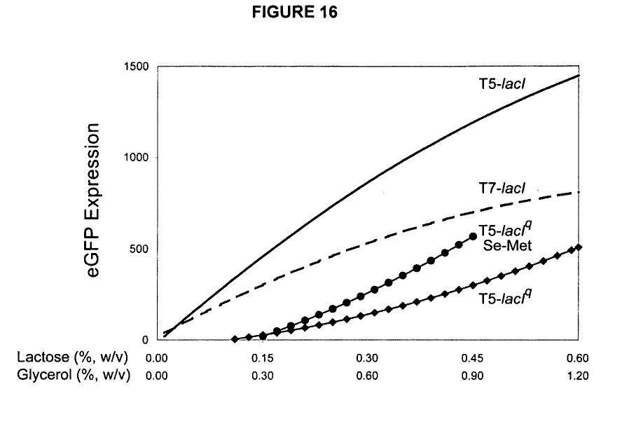

FIG. 16 is a graph showing comparison of modeled expression levels for T5-lacI (solid line), T7-lacI (pET32, dashed line), T5-lacI^{q} in methionine auto-induction medium (filled diamonds) and T5-lacI^{q} in selenomethionine auto-induction medium (filled circles). This figure is a two-dimensional plane through the response surfaces of FIG. 9A (T5-lacI), 9C (T5-lacI^{q}, methionine medium), 9D (T5-lacI^{q}, selenomethionine medium) and 3E (T7-lacI, pET32) starting from zero glycerol and lactose and ending at 1.2% (w/v) glycerol and 0.6% (w/v) lactose, a trajectory that includes the highest response for all cases. This simplified representation offers a direct comparison of expression results achieved from the three expression systems.

As shown in FIG. 16, expression from T5-lacI (solid line) was higher than from T7-lacI (pET32, dashed line) at all compositions except at the lowest lactose concentrations, where basal expression from T7-lacI was higher (Table 4). T5-lacI^{q} (diamonds, methionine medium; circles, selenomethionine medium) exhibited the lowest expression levels. Surprisingly, the combination of T5-lacI^{q} with selenomethionine medium gave a higher level of expression than the same plasmid with methionine medium, and selenomethionine-labeled protein was obtained with yield of ˜500 μg/mL. This enhanced performance occurred because the presence of selenomethionine shifted the maximal lactose consumption to lower cell density, allowing more complete execution of the auto-induction program.

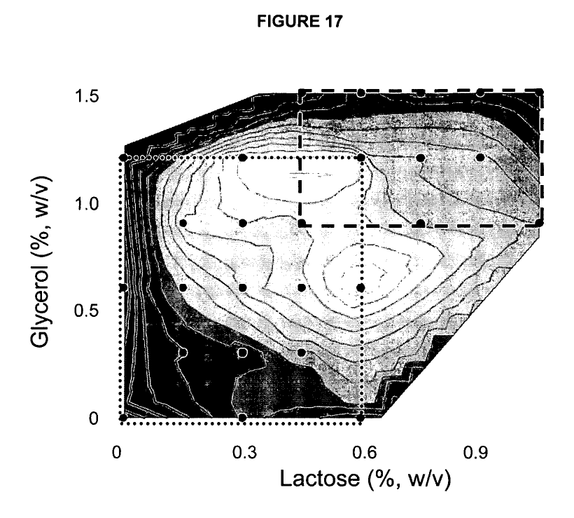

FIG. 17 shows a two-dimensional surface plot that reveals additional features about the composition of the optimal medium for the T5-lacI plasmid. For this plot, the range of carbon source concentrations investigated was intentionally extended beyond that shown in FIG. 9, and resulted in medium compositions that decreased the expression. Lower expression is indicated with blue hues in the original (dark) and higher expression with yellow hues in the original (light). Experimental design points are shown as black circles. The design space explored in the first, lower concentration study is surrounded by dotted lines. For this experiment, a second factorial was completed at higher concentrations of glycerol and lactose for T5-lacI-Luc with methionine media. Dashed lines surround the second factorial, which covers higher concentrations of lactose and glycerol. The contour plot shown here represents a quadratic spline fit to the experimental data, as a single low order model could not adequately model the results due to multiple curvatures. Some fine features of the surface contain experimental uncertainty (such as the “valley” between the two highest expression regions) that would be smoothed out in the response surface models.

The results in FIG. 17 indicate that with the present composition of non-carbon source components, maximum expression from the T5-lacI plasmid is obtained near the limits of the lower factorial (dotted line), specifically 0.6% lactose and 1.2% glycerol and that slightly lower glycerol or lactose concentrations have little effect in this region while higher concentrations of glycerol adversely affect expression (region bounded by the dashed line). The region where highest expression occurred is a broad plateau, indicating overall tolerance to minor variations in medium composition without altering the expression outcome. This plot also shows that there are choices for change in medium composition that give gradual change between lower and higher expression levels. In certain embodiments of the present invention, knowledge of this may be useful to maximize the soluble production of some proteins like luciferase that apparently have an intrinsic solubility limit within cells. Other choices for change in medium composition give precipitous changes in the expression level. In some embodiments of the present invention, knowledge of these is important to avoid experimental conditions that are likely to give poor or irreproducible results.

FIG. 17 also shows that additional increases in lactose and glycerol near the high end of the experimental range investigated did not increase expression, but in some circumstances actually decreased expression. In the present media, the cell density appeared to be limited to OD₆₀₀˜25 and was not affected by further increases in lactose or glycerol, suggesting that some non-carbon source component may have become limiting at this cell density. Systematic evaluation of the contribution of other media components to expression results in a manner similar to that used here for carbon sources may yield further increases in cell density and volumetric protein expression.

Glucose always was the preferred carbon source. Thus, changes in the level of glucose added to the medium control the cell density at which the auto-induction protocol will be initiated. Increasing the level of glucose will increase the cell mass and biological demand for carbon sources, leading to more rapid consumption of lactose and glycerol during the auto-induction phase without compensating changes in the levels of lactose and glycerol. This would shorten the time of auto-induction. Depending on circumstances, this may be beneficial or not.

### Influence of LacI on Auto-induction

LacI acts in two ways to delay the onset of lactose consumption required for auto-induction. First, high intracellular concentrations of LacI increase the occupancy of the lacO sites located upstream of the lac operon structural genes. This occupancy strongly decreases the basal expression of β-galactosidase and lac permease, which in turn decreases the rate of allolactose production. Second, a larger absolute amount of allolactose is required in order to dissociate intracellular LacI from lacO sites so that induction of the lac operon and heterologous protein expression can begin. These combined effects are sufficiently dominant to completely change the order of carbon source consumption from glucose/lactose/glycerol to glucose/glycerol/lactose for E. coli growths with each of the plasmids tested that supplement LacI expression.

Maximal lactose consumption occurred at a higher cell density for the growths with the T5-lacI plasmid (FIG. 14) as compared with the T7-lacI (pET32) plasmid. Since both plasmids have the pBR322 origin, the copy number should not differ greatly. Moreover, since both use the lad promoter to express LacI from the plasmid, the level of LacI should be similar. Small differences in LacI expression due to positional effects in the plasmid may account for the difference in behavior. Positional effects can influence basal levels of heterologous protein expression and it is plausible that positional effects could influence constitutive expression of LacI in a similar way.

It is not clear why expression levels from the T5-lacI plasmid were ˜70% higher than those determined for the pET32 plasmid (T7-lacI, compare FIGS. 9A and 9C). The T5 promoter uses E. coli RNA polymerase, while pET32 requires that T7 RNA polymerase must also be made. It seems unlikely that this difference alone accounts for the lower expression from the pET32 plasmid. T7 RNA polymerase is highly active and might be expected to make more mRNA than E. coli polymerase, especially upon considering that the T7 polymerase is dedicated to the production of target gene transcripts while the T5 promoter must compete with other host promoters. It is possible that high transcription levels may excessively direct energy fluxes towards mRNA production and away from protein expression. Furthermore, transcript instability due to a decoupling of transcription and translation caused by the high transcription rate of T7 RNA polymerase may play a role.

### Influence of Oxygenation State on Auto-Induction

The consequence of oxygenation state in the auto-induction culture is apparent from FIG. 15. In all cases investigated, lactose consumption and protein expression were shifted to a lower cell density by O₂-limitation. For T5-lacI^{q}, this effect was dramatic enough that the final expression levels were higher when O₂ was limited. E. coli is known to control glucose and lactose import as a response to O₂-limitation through a variety of transcriptional and post-translational mechanisms. As one example, phosphorylated ArcA is a negative transcriptional regulator of the IICB^{Glc} component of the bacterial phosphoenolpyruvate:sugar phosphotransferase (PTS) system. Decreased expression of IICB^{Glc} leads to an accumulation of the phosphorylated PTS enzyme component IIA^{Glc}. Since dephosphorylated IIA^{Glc} is a known inhibitor of lac permease, O₂-limitation and accumulation of phosphorylated IIA^{Glc} relieve the inhibition of lac permease, allowing higher lactose import rates.

The results elucidate the origin of the difference in expression behavior observed between small- and large-scale experiments. In the tests of FIG. 7A, the elevated level of LacI postponed lactose utilization in the aerobic conditions of the small-scale, while the O₂-limited conditions of the large-scale shifted lactose utilization to lower cell density and thus promoted protein expression. Reformulation of the carbon sources promoted growth to higher cell density, utilization of lactose at lower cell density, and more complete utilization of provided carbon sources, regardless of the culture oxygenation state (FIG. 7B).

### Role in High-Throughput Protein Expression Studies

For high throughput studies, it is often desirable to screen for suitable protein expression in small volumes using multi-well growth blocks. This expression environment was found to be aerobic, but surprisingly, led to significantly lower protein expression in the initially defined auto-induction medium. In contrast, O₂-limitation was previously noted to increase the yield of recombinant protein and this limitation most closely corresponds to the actual conditions in 2-L bottles used for large-scale protein expression. It is noted that fully anaerobic conditions are not conducive to either rapid cell growth or high-yield production of biomass.

It may also be desirable to provide for improved correlation between small-scale protein expression screening and large-scale protein production at the University of Wisconsin Center for Eukaryotic Structural Genomics. Through an initial set of media optimization experiments, the discrepancy between small and large scale culture results was addressed by increasing the concentration of carbon sources available, which on average gave a 2- to 3-fold increase in target protein expression in the small-scale cultures. However, protein expression in large-scale cultures was only marginally improved with this initial change in medium composition. Therefore, other manipulations of the biochemical apparatus used for auto-induction were tested for improvement of protein expression yields. By decreasing the LacI expression level provided by the expression plasmid, lactose consumption and heterologous protein expression were shifted to an earlier phase of growth. Although culture oxygenation still contributed to the timing of induction, both small- and large-scale growths were able to produce enough allolactose to derepress the lac operon, fully consume the available lactose, and achieve high levels of protein expression.

These studies also give insight into the use of auto-induction for production of ¹³C- and ¹⁵N-labeled proteins for NMR structure determination. First, efficient consumption of succinate during the auto-induction process limits the utility of these media formulations for production of ¹³C-labeled samples for NMR structure determination, unless ¹³C-labeled succinate is used. The substitution of other amino acids (aspartate, glutamate) will not correct this problem and potentially introduce problematic dilution of ¹⁵N-labeling unless the ¹⁵N-labeled analogs are used. Furthermore, the previously described changes in medium composition for NMR studies (Tyler et al., 2005, Protein Expr. Purif. 40: 268-278) are now recognized to fall into a low productivity region of the T5-lacI^{q} response surface shown in FIG. 9B (0.5% glycerol, 0.2% lactose). In the previous study (Tyler et al., 2005), unlabeled lactose (not cost-effective for use as a ¹³C-labeled compound) was intentionally minimized in order to avoid isotopic dilution of ¹³C-labeled glycerol. As an alternative, the response curve in FIG. 9A suggests this same mixture of glycerol and lactose may give considerably better expression results when coupled with a T5-lacI plasmid.

### Other Possible Uses of Factorial Medium Design

Small-scale protein expression in 24- or 96-well blocks was originally intended to be a screening tool for numerous structural genomics targets, whose expression properties were not known. The array format for the various auto-induction media provides a simple way to test the performance of other plasmid vectors and host strains for conditions that maximize the expression of these unknown proteins. Moreover, based on expression levels possibly exceeding 1000 μg/mL of culture fluid (actually exceeding 2000 μg/mL for the combination of MBP and eGFP from pVP62K), it is reasonable to consider other applications for small-scale expression with known proteins. For example, the amount of protein produced from a few mL of these cultures may be sufficient for automated protein purification, microfluidics-based crystallization screening, initial nL-scale crystallization trials, ¹⁵N HSQC NMR measurements, or functional and enzymatic characterizations.

Other Experience with Use of Optimized Auto-Induction Conditions

The work presented here includes expression studies in 96-well growth blocks, 2-L shaken bottles and automated stirred-vessel fermenters. In each case, the combination of a designed auto-induction medium and matched expression plasmid gave strong expression results, demonstrating utility in several different formats used to grow bacterial cells. The results presented here derive from study of two target proteins, eGFP and luciferase, that were chosen due to the attractiveness of their assays. Nevertheless, the experience with other proteins suggests that these modifications to auto-induction media composition and LacI dosing may have general utility in improving the level of recombinant protein expression. Thus, combination of a T5-lacI expression plasmid with a terrific broth medium supplemented with an auto-induction mixture of 0.015% glucose, 0.8% glycerol, 0.5% lactose, 0.375% aspartic acid and 2 mM MgSO₄ contributed to a ˜5-fold increase in expression of soluble TEV protease when compared to previous reports. Moreover, expression studies with other proteins such as toluene 4-monooxygenase hydroxylase, stearoyl-ACP Δ9 desaturase, cytochrome b₅, mouse Rieske ferredoxin, and various bacterial and plant FMN-containing oxidoreductases, indicate the combination of a factorial designed auto-induction media with T5-lacI plasmids offers substantial promise for structural and functional work with known proteins.

### Example of Improved Performance of Auto-Induction Medium Through Empirical Experiments

In this example, the performance of the auto-induction medium was modified and improved through empirical experiments. The object was to define conditions that would give consistent screening results, regardless of the expression scale, so as to increase the predictive reliability of small-scale screening. As a starting point, the large difference observed in the cell density achieved at saturation in small-scale auto-induction experiments (OD₆₀₀˜20-25) and small-scale defined medium experiments (OD₆₀₀˜10) was considered. To determine if the problem was due to limitation in carbon sources, the relationship between protein expression levels and the concentration of glucose, glycerol, lactose and aspartic acid or succinic acid was investigated using a factorial design approach.

Upon variation of the concentrations of carbon sources, the corresponding changes in protein expression were determined by assay of two proteins expressed from the T5/lac2 and T7/lac expression systems: green fluorescent protein (GFP) and human rhinovirus 14 3C protease (3CP). After determination of the protein expression levels, a new center point (FIG. 1, dark sphere) was chosen based on the best previous result and the factorial process was continued.

Values of the Cᵢ obtained from a factorial experiment for glucose and lactose are shown in Table 6. These representative values associate the percent change of GFP fluorescence derived from a 1% change in (w/v) of the two carbon sources. For example, in the T5/lac2 system, a 0.1% increase in glucose concentration would cause about 50% decrease in GFP expression. Likewise, a 0.1% increase in lactose concentration would cause about 30% increase in GFP expression. These values can only be considered valid within the range of independent variables evaluated. The response coefficients for other constituents are separately defined. For example, the response coefficients for glycerol and aspartic acid were evaluated in other, separate experiments.

TABLE 6
Response of GFP expression to changes in glucose and lactose
concentrations around a midpoint of 0.05% glucose and 0.2% lactose

| Values of Cᵢ | T5/lac2 | T7/lac |
|---|---|---|
| Glucose | −660 ± 130 | −900 ± 350 |
| Lactose | 288 ± 37 | 104 ± 100 |

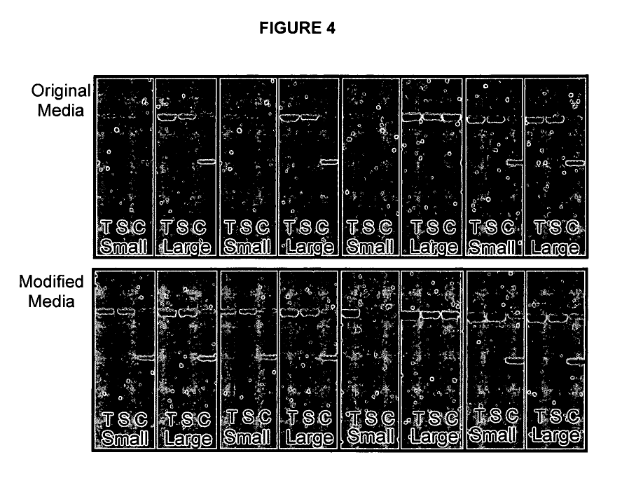

In some experiments, a T5/lac2 expression vector pVP27 was used. This vector has a high level of LacI expression, has relatively low basal expression, and does not require T7 RNA polymerase. The media modification for improved T5/lac2 expression included decrease in the amount of glucose from 0.05% to 0.15% (w/v); increase in the amount of lactose from 0.2% to 0.5% (w/v); increase in the amount of glycerol from 0.5% to 0.8% (v/v); and an increase in the amount of dicarboxylic acid from 0.25% to 0.375% (v/v). In this experiment, approximately 60 media formulations were tested, using four different expression targets, and the protein expression data are shown in FIG. 4.

The top panels in FIG. 4 show the initial auto-induction expression results of small-scale screening and large-scale production conducted in a defined original auto-induction medium containing 0.05% glucose, 0.2% lactose, 0.5% glycerol, and 0.25% aspartic acid (Studier, 2005, Protein Expr. Purif. 41: 207-234). The gels were imaged after reaction of the fluorophore FlAsH with the tetra cysteine (C4) motif incorporated into the fusion protein. The locations of the fusion protein (F) and MBP after TEV cleavage (M) are shown. Expression levels for targets 1-3 were considerably lower for small-scale than for large-scale, while only target 4 exhibited similar expression.

Expression results obtained from modified medium on either small or large-scale for the same four targets are shown in the bottom panels in FIG. 4. The carbon source concentrations in the modified medium were: glucose, 0.015%, lactose, 0.5%, glycerol, 0.8%, aspartic acid, 0.375%. Three of the four targets shown had identical small and large-scale scoring results. The typical correlation for production is ˜80%, as compared to ˜50% correlation with the media used before factorial improvement.

Not wanting to be bound by the following theory, it is possible that higher concentrations of glycerol and dicarboxylic acid lead to higher cell densities, resulting in oxygen limitation for small scale cultures. Oxygen limitation promotes lactose consumption. Lactose, as primary remaining energy source during induction, promotes higher levels of protein production. The oxygen limitation in large scale culture with original media is less sensitive to media modification.

### Improved Large-Scale Production of Tobacco Etch Protease

Tobacco etch virus Nla proteinase (TEV protease) is an important tool for the removal of fusion tags from recombinant proteins. Production of TEV protease in E. coli has been hampered by insolubility and addressed by many different strategies. Using an engineered TEV protease lacking the C-terminal residues 238-242 and the methods of the present invention, expression of TEV protease at high levels and with high solubility was obtained by using auto-induction medium at 37° C. In combination with the expression work, an automated two-step purification protocol was developed that yielded His-tagged TEV protease with >99% purity, high catalytic activity and purified yields of ˜400 mg/L of expression culture (˜15 mg pure TEV protease per g of E. coli cell paste). Methods for producing glutathione S-transferase (GST) tagged TEV with similar yields (˜12 mg pure protease fusion per g of E. coli cell paste) are also reported.

TEV Protease Expression Vectors. The expression vector pQE30-S219V containing a TEV protease gene was obtained from Prof. B. F. Volkman and Dr. F. Peterson at the Medical College of Wisconsin (Milwaukee, Wis.). This pQE30-derived plasmid (Qiagen) encoded residues 1-242 of the TEV protease open reading frame, the native residues at the C-terminus and the S219V mutation, which conferred resistance to auto-inactivation. The expression vector pQE30-S219VpR₅ was a variant of pQE30-S219V where residues 238-242 were each replaced with arginine residues to create a poly-Arg₅ tag (pR₅) at the C-terminus. The expression vector pRK793 encoding a self-cleaving MBP-His₇-TEV-pR₅ protease fusion protein was obtained from Dr. D. S. Waugh at the National Cancer Institute (Frederick, Md.). pRK793 also encoded the S219V mutation. The MBP-His₇-TEV-pR₅ fusion can undergo proteolysis in vivo at a TEV protease site in the linker region after MBP to liberate MBP and His₇-TEV-pR₅.

Using standard molecular biology methods, PCR primers were used to prepare TEV protease variants by overlap extension PCR. All DNA fragments prepared by PCR amplification were sequence verified. The solubility enhancing mutations T17S, N68D, and 177V described previously were incorporated into certain TEV protease variants as indicated below. Separate PCR reactions were used to generate three fragments, one consisting of the N-terminus through T17S, a second between T17S and N68D/177V, and a third between N68D/177V and the desired C-terminus.

The PCR primers for the 5′ fragments were designed to produce protein with an N-terminal His₇-tag (TEV-For-H7) or protein with no N-terminal tag (TEV-For-NoTag). The 5′ fragment primers also contained the SgfI restriction site for Flexi vector cloning. The PCR primers for the central fragment duplicated the gene from the solubility enhancing mutation T17S (T17S-For) to the other mutations N68D/177V (N68D-177V-Rev). The PCR primers for the 3′ fragments C-terminal fragments were designed to produce protein with different C-terminal extensions. The reverse primers also encoded the PmeI restriction site for use in Flexi vector cloning. The primers N68D-177-For and TEV-Rev-Full were used to generate a full-length 242-residue TEV protease. The TEV protease was also truncated at either residue 238 (protein designated 238Δ, using primers N68D-177-For and TEV-Rev-L239) or at residue 233 (233Δ, using primers N68D-177-For and TEV-Rev-L234). The complete coding region was assembled from these fragments by a second round of PCR.

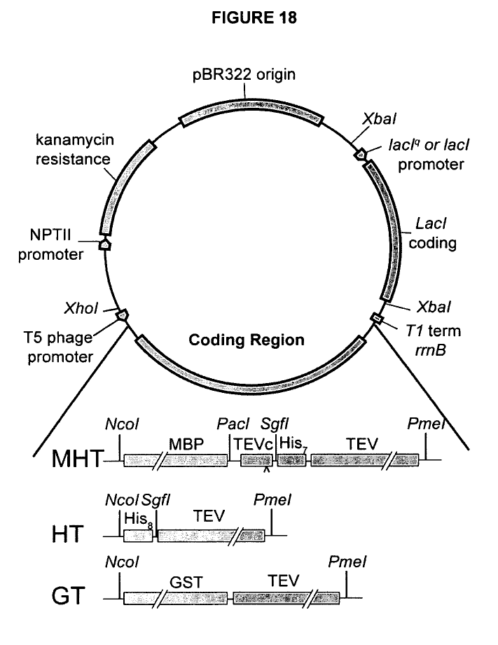

Vector maps. FIG. 18 illustrates maps of three expression vectors used. PCR products were incorporated into these expression vectors either directly from the overlap PCR or by transfer from another Flexi vector. The vectors are identical except for the coding region and the promoter used for expression of LacI. The MHT coding region produces MBP-His₇-TEV with a TEV protease site (TEVc) between MBP and the His₇ sequence. After cleavage at the TEVc site, the MHT coding region yields Ala-Ile-Ala-His₇-TEV. The HT coding region yields His₈-TEV. The GT coding region produces a non-cleavable GST-Leu-IleAla-TEV protease fusion with no His-tag. Expression levels from auto-induction were increased by replacing the lacI, promoter with a wild type lad promoter in some of the vectors.

Expression Hosts. Escherichia coli BL21 (EMD Biosciences/Novagen), E. coli BL21 RILP (Stratagene), and E. coli Krx (Promega) were used as expression hosts. The RILP strain contains a plasmid for codon adaptation that provides constitutive expression of several tRNAs that are in low abundance in E. coli, including argU previously found to be important for TEV expression.

TEV Protease Expression. Expression studies were carried out using either auto-induction (Sreenath et al., 2005, Protein Expres. Purif. 40: 256-267; Studier, 2005, Protein Expr. Purif. 41: 207-234) or isopropylthio-galactoside (IPTG) induction. Kanamycin (100 μg/mL) was added to all media and chloramphenicol (34 μg/mL) was added to cultures of E. coli BL21 RILP. All starting inocula were grown in chemically defined MDAG medium (Studier, 2005, Protein Expr. Purif. 41: 207-234) modified by the addition of 0.375% aspartic acid, 0.8% glucose, and reduction of phosphate to 25 mM. Starting inocula were grown overnight at 25° C. and reached saturation at OD₆₀₀ of ˜10 to 15. The starting inoculum was added at 1/20^{th} the volume of expression medium. Expression medium consisted of terrific broth containing 0.8% glycerol (Sigma, St. Louis, Mo.) prepared according to the manufacturer's instructions and further supplemented with 2 mM MgSO₄ and 0.375% aspartic acid. When used for induction, IPTG was added to a final concentration of 0.5 mM. For auto-induction, the medium also contained 0.5% (w/v) lactose and 0.015% (w/v) glucose.

Small-scale expression screening was conducted in 96-well growth blocks (Qiagen) containing 400 μL of medium. For IPTG induction, the cultures either were grown at 37° C. and treated for 3 h with IPTG or were grown at 25° C. and treated for 5 h with IPTG. The IPTG induction was initiated when culture monitoring showed OD₆₀₀≈1.2-2.0, which corresponded to early log phase growth. For auto-induction, the expression screening was carried out for either ˜12 h at 37° C. or ˜24 h at 25° C. No additional monitoring after inoculation was required. The small-scale cultures were harvested by freezing 100 μL aliquots at −80° C.

Large-scale expressions were done either in 2-L PET bottles containing 0.5 L of culture medium or in a Bioflow 3000 fermenter (New Brunswick Scientific, Edison, N.J.) containing 9.5 L of culture medium. The large-scale cultures were pelleted by centrifuge at 4000×g for 20 min. The cell pellets were re-suspended in a small volume of 50 mM phosphate, pH 7.5, containing 300 mM NaCl and 20% ethylene glycol and centrifuged again to recover the washed cell paste. The washed cell paste was stored at −80° C. in 50 mL conical tubes.

Preparation of Small-Scale Cell-Free Lysates. the Cell Cultures frozen in PCR plates were thawed and suspended in lysis buffer to a final volume of 120 μL and a final composition of 20 mM Tris-HCl, pH 7.5, 20 mM NaCl, 0.3 mM (TCEP), 1 mM MgSO₄, 3 KU/mL of rLysozyme (EMD Biosciences/Novagen) and 0.7 U/mL of benzonase (EMD Biosciences/Novagen). After 30 min incubation at room temperature, the samples were sonicated on a plate sonicator (Misonix, Farmingdale, N.Y.) for 6 to 10 min. Samples were then centrifuged at 3000×g for 30 min. The supernatant fraction was retained for protease assay measurements.

TEV Protease Activity Assays. TEV activity was determined using a fluorescence anisotropy based protease assay (Blommel and Fox, 2005, Anal. Biochem. 336: 75-86) with the soluble fraction of the cell-free lysate. The assay is based on a reduction in fluorescence anisotropy that occurs when a small fluorescent peptide is liberated from a larger protein. For this work, the substrate reported earlier was modified to minimize the anisotropy upon proteolysis by minimizing the size of the liberated peptide. This fluorescent substrate was produced in E. coli as the fusion protein His₈-MBP-3CPc-C4-attB1-TEVc-MBP, where His₈ is an N-terminal His-tag that consists of eight Histidine residues, MBP is E. coli maltose binding protein, 3CPc is a human rhinovirus 3C protease cleavage site, LEVLFQ↓GP (SEQ ID NO:4), where ↓ indicates the 3C protease cleavage site; C4 is the tetracys motif, CCPGCC (SEQ ID NO:5), attB1 is the amino acid sequence required for the attB1 site of Gateway cloning, TSLYKKAGS (SEQ ID NO:6) and TEVc is a TEV protease cleavage site, ENLYFQ↓S (SEQ ID NO:7).

The fusion protein was expressed and purified as previously reported. After treatment with 3C protease, the substrate protein (TM3CP) has the N-terminal sequence of GPCCPGCCTSLYKKAGSENLYFQ↓S (SEQ ID NO:8) fused to MBP. FlAsH was synthesized (Adams et al., 2002, J. Am. Chem. Soc. 124: 6063-6076) and added to TM3CP in an amount sufficient to provide ˜5% covalent labeling of the tetracys motif. The standard proteolysis assay was performed in 20 mM Tris, pH 7.5, containing 100 mM NaCl, 5 mM EDTA, 0.3 mM triscarboxyethylphosphine (TCEP) and 5 μM TM3CP with 5% FlAsH labeling at 25° C. to 28° C. Proteolysis releases the fluorescently labeled peptide GPCCPGCCTSLYKKAGSENLYFQ (SEQ ID NO:9). Samples of the fluorescent substrate incubated with TEV protease at conditions known to effect complete cleavage were used to determine the intrinsic anisotropy, mrᵢ, of the peptide in the given assay conditions. The time-dependent exponential changes in fluorescence anisotropy were fit by non-linear least squares methods to determine the initial anisotropy, mr₀, the final anisotropy, mr_{∞} and the decay constant (proteolysis rate). The mr₀, mr_{∞} and mrᵢ values were used to prepare fractional progress curves. Fitted decay constants were adjusted for the percentage labeling of the substrate. Reported errors for the assay represent two standard deviations of the mean.

Refolded TEV Protease. S219V-TEV protease expressed from IPTG-induced cultures of E. coli BL21 pQE30-S219V was prepared by re-suspension of the inclusion bodies in 6 M guanidinium hydrochloride containing 0.3 mM TCEP to a final protein concentration of 1 mg/mL. This suspension was diluted 20-fold into a refolding buffer containing 50 mM MES, pH 6.5, containing 0.5 M arginine, 0.5 M sucrose, 2 mM MgCl₂, and 0.3 mM TCEP. After 1 h, the refolded mixture was subjected to IMAC purification and dialyzed into storage buffer containing 50% glycerol.

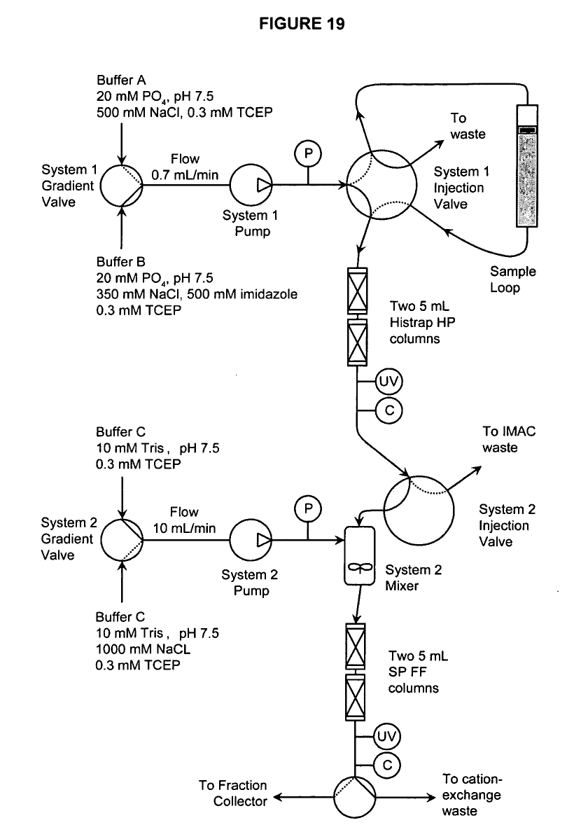

Purification of His-TEV Protease. FIG. 19 shows a schematic of the instrumentation (AKTA set-up) and buffer compositions used for TEV purification. The Akta Prime system and all other equipment and chromatography resins were from GE Healthcare Life Sciences (Piscataway, N.J.). Buffer A was 20 mM phosphate, pH 7.5, containing 500 mM NaCl and 0.3 mM TCEP. Buffer B was 20 mM phosphate, pH 7.5, containing 350 mM NaCl, 500 mM imidazole and 0.3 mM TCEP. Buffer C was 10 mM Tris, pH 7.5, containing 0.3 mM TCEP. Buffer D was 10 mM Tris, pH 7.5, containing 1000 mM NaCl and 0.3 mM TCEP. Control programs were developed to complete consecutive IMAC and cation exchange purifications without user intervention.

Cell paste (34 g) was re-suspended in 50 mM phosphate, pH 7.5, containing 300 mM NaCl, 20% ethylene glycol and 0.3 mM TCEP at a ratio of 6 mL of buffer per g of wet cell paste. The following protease inhibitors were added to the indicated final concentrations prior to sonication: E-64 (1 μM), EDTA (1 mM) and benzamidine (0.5 mM). The cell suspension was sonicated for 6 min on ice and all subsequent purification steps were conducted at 4° C. The sonicated cell suspension was centrifuged for 25 min at 95,000×g and the soluble fraction was retained. The soluble fraction was loaded into either a 50 or 150 mL loading loop and then loaded onto purification system 1 at 3 mL/min. This purifier system had two 5 mL Histrap HP columns arranged in series and equilibrated with buffer A. The columns were washed with eight volumes of a mixture of 85% buffer A and 15% buffer B. During the wash, the flow rate was increased to 5 mL/min.

The bound protease was eluted from purification system 1 by a step-wise change to 100% buffer B. At the start of the elution step, the flow rate of buffer B was decreased to 0.7 mL/min and the flow path was diverted to purification system 2. This purification system had a 2 mL mixing chamber upstream of two 5 mL SP Fast Flow columns arranged in series. The columns were equilibrated with buffer C. The sample from the first purifier was injected into the mixing chamber at 0.7 mL/min, mixed with 100% buffer C at 10 mL/min and loaded onto the columns of purification system 2 at a total flow rate of 10 mL/min. The resultant ˜15-fold dilution of the sample prior to application to the cation exchange columns ensured that the ionic strength was low enough allow tight binding of the protease to the column.

Upon completion of the IMAC elution, the flow through purifier system 1 was increased to 5 mL/min and directed to waste for column wash and re-equilibration with buffer A prior to the injection of the next aliquot of lysate. The waste sample was collected so that possible losses of TEV protease could be determined. Upon completion of the IMAC elution, the flow through purifier system 2 was decreased to 5 mL/min and a six column volume gradient from 100% buffer C to a mixture of 40% buffer C and 60% buffer D was started. Fractions containing TEV protease were detected by UV measurement. After elution of the TEV protease, the flow through purification system 2 was directed to waste. The column was then washed with several volumes of 100% buffer D and re-equilibrated with 100% buffer C prior to the start of the next injection from the first purification system. This waste sample was also collected.

Fractions were analyzed by catalytic assays and SDS-PAGE and were pooled based on specific activity and protein purity. The protein concentration of the pooled sample was determined by UV-visible spectroscopy (λ₂₈₀=32770 calculated from the amino acid composition). The pooled TEV protease was diluted with buffer C and storage buffer containing 10 mM Tris, 0.5 mM EDTA, 0.3 mM TCEP and 80% (v/v) glycerol to a protein concentration of 1 mg/mL in 50% glycerol. No additional buffer exchange, concentration or dialysis steps were required. The purified TEV protease was stored in this buffer at −20° C.

Purification of GST-TEV Protease. For purification of GST-TEV, the preparation of the cell-free lysate and soluble fraction from 3 g of cell paste were as described above. Ammonium sulfate was added to 55% of saturation in order to precipitate the protease fusion. The pellet from the ammonium sulfate precipitation was re-suspended in 20 mL of 10 mM Tris, pH 7.5, containing 10 mM NaCl and 0.3 mM TCEP. The glutathione sepharose purification step was completed using an 8 mL gravity flow column at room temperature because the GST-TEV was found to bind slowly to the resin at 4° C. The column was washed with five column volumes of the re-suspension buffer described above. The protein was eluted with 50 mM Tris, pH 7.5, containing 2 mM EDTA, 0.3 mM TCEP and 10 mM reduced glutathione. The eluted fusion protein was concentrated using an Amicon 10 kDa molecular weight cutoff centrifugal concentrator (Millipore, Billerica, Mass.) to a concentration of ˜18 mg/mL. The concentrated sample was loaded to a Sephacryl S-100 26/10 column equilibrated in 10 mM Tris, pH 7.5, containing 1 mM EDTA and 0.3 mM TCEP at 4° C. at a flow rate of 1 mL/min. Fractions were analyzed as described above.

FIG. 19 is a schematic representation of the equipment used for automated two-step purification of His₇-TEV protease. The solid lines in the system injection valves show the flow path during the sequential IMAC elution and cation exchange binding phase of the purification. The dotted lines indicate flow paths used during other phases of the purification. Separate control programs were developed for the IMAC and cation exchange steps and were synchronized by starting the programs at the same time. By specifying the timing of steps that require coordinated action of both units, no communication between the purification units was required. Abbreviations: P, pressure sensor; UV, absorbance detector making measurements at 280 nm; C, conductivity detector.

Other Analytical Methods. Protein expression levels were assessed using SDS-PAGE on total cell lysates, and the soluble and insoluble fractions prepared as previously reported (Sreenath et al., 2005, Protein Expres. Purif. 40: 256-267). The molecular weight markers shown in gels were from BioRad (Hercules, Calif.). Mass spectral analyses were determined using a Sciex API 365 triple quadrupole mass spectrometer (Perkin Elmer, Boston, Mass.) maintained at the University of Wisconsin Biotechnology Center.

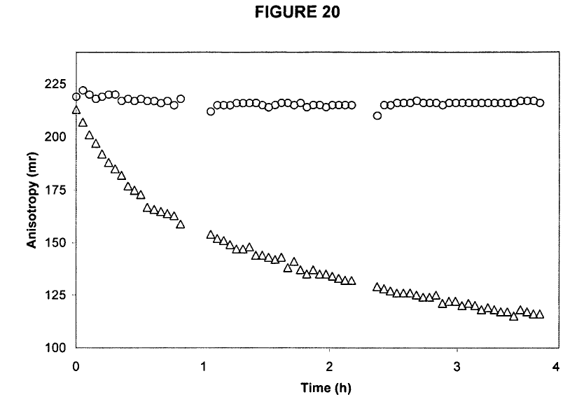

FIG. 20 is a representative fluorescence polarization assay of TEV protease activity present in an E. coli cell lysate. Open circles show anisotropy data for an E. coli lysate that did not contain TEV protease. Open triangles show results from expression of MHT238Δ. The gaps in the data occurred when the assay plate was removed from the instrument to add components for additional assays in other wells.

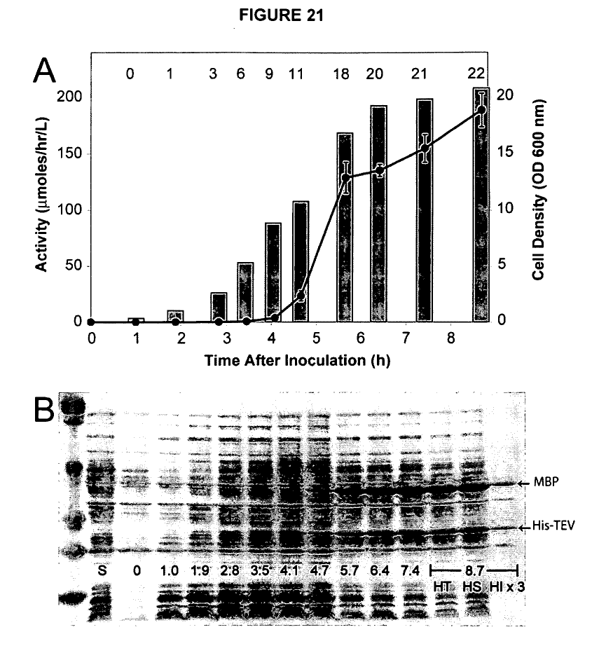

The highest level of TEV protease might be produced from MHT238 at 37° C. using auto-induction, RILP codon adaptation and the lad promoter for regulation of LacI expression. FIG. 21 shows results from the expression of TEV protease during auto-induction from MHT238Δ in a 10-L fermenter. FIG. 21A shows the time course of changes in TEV protease activity and cell density, and the correlation of TEV protease activity and cell density with duration of the fermentation. Error bars for the activity measurements represent two standard deviations above and below the mean. Cell densities are shown as bars and as numbers across the top of the plot. During the auto-induction process, the TEV protease activity was below detection limits until the cell density reached ˜6 (3.5 h after inoculation). Thereafter the protease activity increased rapidly with the largest increase occurring between cell densities of 10 and 18 (5 to 7 h after inoculation). FIG. 21B shows an SDS-PAGE gel analysis of the expression culture. The SDS-PAGE results are consistent with the assay results, as the protein bands corresponding to both MBP and His-TEV appeared ˜4.7 h after induction. Expressed MBP-His₇-TEV238Δ fusion protein is cleaved during cell growth to separate MBP and His₇-TEV238Δ. Arrows indicate the position of MBP and His₇-TEV after in vivo cleavage. The lane marked S contains a sample of the starting inoculum grown in a non-inducing medium. The lanes marked with time correspond to the data points indicated in A. The lanes marked HT, HS, and HI are the total, soluble, and insoluble fractions obtained at harvest, 8.7 h after inoculation. The amount of sample loaded was normalized by cell density for all lanes except the insoluble harvest sample, which was loaded at 3× the normalized amount to allow better visualization. The cells were harvested after ˜9 h, yielding 23 g of wet cell paste per liter of culture medium (total 230 g of cell paste). The rightmost three lanes in FIG. 21B show that the TEV protease was almost exclusively soluble, with less than 5% of the protease accumulated in the insoluble fraction based on scanning densitometry (note that the insoluble fraction was loaded at 3× the equivalent volume in the SDS-PAGE to allow better visibility).

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of biochemistry, growth media, and protein expression, are within the scope of this invention. All publications, patents, and patent applications cited herein are incorporated by reference in their entirety for all purposes.

What is claimed is:

1. A method for designing a culture medium that promotes induction of transcription of heterologous DNA in cultures of bacterial cells, comprising:
a) providing a bacterial cell comprising a recombinant expression vector comprising the heterologous DNA operably connected to a promoter whose activity can be induced by one or more constituents of the culture medium;
b) defining a first medium constituent;
c) changing the concentration of the medium constituent in the culture medium;
d) growing the bacterial cell in the culture medium to express the heterologous DNA;
e) evaluating the outcome of the change in concentration of the medium constituent to determine the change that gives the most favorable result for expression of heterologous DNA;
f) adopting the changed concentration of the medium constituent that gives the most favorable result as a new starting condition for the culture medium;
g) defining a next medium constituent; and
h) repeating steps c) to e) with the next medium constituent of the culture medium to determine an improved composition of the culture medium for promoting transcription of the heterologous DNA.

2. The method of claim 1 wherein changing the concentration of the medium constituent comprises increasing or decreasing the concentration of the medium constituent in the culture medium.

3. The method of claim 1 wherein the first medium constituent and the next medium constituent comprise carbon sources.

4. The method of claim 1 wherein the first medium constituent and the next medium constituent comprise carbon sources selected from one or more of the group consisting of glucose, lactose, glycerol, rhamnose, arabinose, succinate, fumarate, malate, citrate, acetate, maltose and sorbitol.

5. The method of claim 1 wherein the medium constituent comprises a pH buffering compound.

6. The method of claim 5 wherein the pH buffering compound comprises a dicarboxylic acid selected from one or more of the group consisting of oxalic acid, aspartic acid, fumaric acid, glutamic acid, succinic acid, malonic acid, glutaric acid, and phthalic acid.

7. The method of claim 1 wherein the bacterial cell is an Escherichia coli cell.

8. The method of claim 1 wherein the cells are grown batchwise.

9. The method of claim 1 wherein the promoter is selected from the group consisting of a lac promoter, a T7 promoter, a T7/lac promoter, a T5 promoter, or a T5/lac promoter.

10. The method of claim 1 wherein the promoter is repressed by a lac repressor.

11. The method of claim 1 wherein the culture medium comprises from about 0.01% w/v to about 0.02% w/v of glucose.

12. The method of claim 1 wherein the culture medium comprises from about 0.4% w/v to about 0.6% w/v of lactose.

13. The method of claim 1 wherein the culture medium comprises from about 0.7% w/v to about 0.9% w/v of glycerol.

14. The method of claim 1 wherein the culture medium comprises from about 0.35% w/v to about 0.40% w/v of dicarboxylic acid.

15. The method of claim 1 wherein the culture medium comprises about 0.001% w/v to about 0.5% w/v of glucose, about 0.01% w/v to about 3% w/v of lactose, and about 0.1% w/v to about 5% w/v of glycerol.

16. The method of claim 15 wherein the culture medium further comprises about 0.05% w/v to about 4% w/v of dicarboxylic acid.

17. The method of claim 1 wherein the culture medium comprises about 0.01% w/v to about 0.02% w/v of glucose, about 0.4% w/v to about 0.6% w/v of lactose, and about 0.7% w/v to about 0.9% w/v of glycerol.

18. The method of claim 17 wherein the culture medium further comprises about 0.05% w/v to about 4% w/v of dicarboxylic acid.

19. A culture medium comprising about 0.001% w/v to about 0.5% w/v of glucose, about 0.01% w/v to about 3% w/v of lactose, and about 0.1% w/v to about 5% w/v of glycerol.

20. The culture medium of claim 19 further comprising about 0.05% w/v to about 4% w/v of dicarboxylic acid.

21. The culture medium of claim 19 comprising about 0.01% w/v to about 0.02% w/v of glucose, about 0.4% w/v to about 0.6% w/v of lactose, and about 0.7% w/v to about 0.9% w/v of glycerol.

22. The culture medium of claim 19 comprising about 0.35% w/v to about 0.40% w/v of dicarboxylic acid.

23. The culture medium of claim 19 comprising about 0.015% w/v of glucose, about 0.5% w/v of lactose, and about 0.8% w/v of glycerol.

24. The culture medium of claim 23 further comprising about 0.375% w/v of dicarboxylic acid.

25. A method for promoting auto-induction of transcription of heterologous DNA in cultures of bacterial cells, comprising:
a) providing a bacterial cell comprising a recombinant expression vector comprising heterologous DNA operably connected to a promoter whose activity can be induced by an exogenous inducer;
b) providing a culture medium comprising about 0.001% w/v to about 0.5% w/v of glucose, about 0.01% w/v to about 3% w/v of lactose, and about 0.1% w/v to about 5% w/v of glycerol; and
c) growing the bacterial cell in the culture medium to express the heterologous DNA.

26. The method of claim 25 wherein the culture medium further comprises a pH buffering compound.

27. The method of claim 26 wherein the pH buffering compound comprises a dicarboxylic acid selected from one or more of the group consisting of oxalic acid, aspartic acid, fumaric acid, glutamic acid, succinic acid, malonic acid, glutaric acid, and phthalic acid.

28. The method of claim 25 wherein the culture medium further comprises between about 0.05% w/v and about 4% w/v of dicarboxylic acid.

29. The method of claim 25 wherein the bacterial cell is an Escherichia coli cell.

30. The method of claim 25 wherein the bacterial cells are grown batchwise.

31. The method of claim 25 wherein the promoter is selected from the group consisting of a lac promoter, a T7 promoter, a T7/lac promoter, a T5 promoter, or a T5/lac promoter.

32. The method of claim 25 wherein the promoter is repressed by a lac repressor.

33. The method of claim 25 wherein the culture medium comprises from about 0.01% w/v to about 0.02% w/v of glucose.

34. The method of claim 25 wherein the culture medium comprises from about 0.4% w/v to about 0.6% w/v of lactose.

35. The method of claim 25 wherein the culture medium comprises from about 0.7% w/v to about 0.9% w/v of glycerol.
